(19) United States
(12) Patent Application Publication
(10) Pub. No.: US 2024/0000839 A1
(43) Pub. Date: Jan. 4, 2024

(54) BCMA-TARGETED CHIMERIC ANTIGEN RECEPTORS

(71) Applicant: SHANGHAI CELLULAR BIOPHARMACEUTICAL GROUP LTD., Shanghai (CN)

(72) Inventors: Yihong YAO, Rockville, MD (US); Jiaqi HUANG, Rockville, MD (US); Xin YAO, Rockville, MD (US)

(21) Appl. No.: 18/255,514

(22) PCT Filed: Dec. 1, 2021
(86) PCT No.: PCT/US21/61410

§ 371 Date: Jun. 1, 2023

Related U.S. Application Data
(63) Continuation-in-part of application No. 17/476,661, filed on Sep. 16, 2021, now Pat. No. 11,498,973.
(60) Provisional application No. 63/212,289, filed on Jun. 18, 2021.
(60) Provisional application No. 63/153,666, filed on Feb. 25, 2021.
(60) Provisional application No. 63/120,692, filed on Dec. 2, 2020.

(51) Int. Cl. A61K 35/17; C07K 16/28; A61K 39/00; A61P 35/00; A61K 9/00

(52) U.S. Cl. CPC A61K 35/17; C07K 16/2878; A61K 39/4611; A61K 39/4613; A61K 39/4631; A61K 39/464417; A61P 35/00; A61K 9/0019; A61K 2239/17; A61K 2239/21; A61K 2239/13; A61K 2239/48

(57) ABSTRACT

The present disclosure provides BCMA-targeted chimeric antigen receptors (CARs) as well as preparation methods and applications thereof. The CARs of the present disclosure targets BCMA-positive cells, and can be used for treating BCMA-positive B-cell lymphoma, multiple myeloma and plasma cell leukemia.

### CROSS REFERENCE TO RELATED APPLICATIONS

The present application claims priority to U.S. Provisional Application Nos. 63/120,692 (filed on Dec. 2, 2020), 63/153,666 (filed on Feb. 25, 2021), and 63/212,289 (filed on Jun. 18, 2021), and U.S. application Ser. No. 17/476,661 (filed on Sep. 16, 2021), each of which is hereby incorporated by reference in its entirety.

### SEQUENCE LISTING

This application contains a Sequence Listing which has been filed electronically in ASCII format and is hereby incorporated by reference in its entirety. Said ASCII copy, created on Dec. 1, 2021, is named 11299-008884-WO1_ST25.txt and is 41 KB in size.

### TECHNICAL FIELD

The invention relates to the field of biomedicine, and more particularly to chimeric antigen receptors targeting BCMA, as well as preparation methods and applications thereof.

### BACKGROUND

BCMA is a B cell maturation antigen, also known as CD269 or TNFRSF17, and is a member of the tumor necrosis factor receptor superfamily. Its ligands are B cell activating factor (BAFF) and a proliferation-induced ligand (APRIL).

Binding of BCMA to BAFF and APRIL activates NF-kB and induces up-regulation of anti-apoptotic Bc1-2 members such as Bc1-xL or Bc1-2 and Mc1-1. The interaction between BCMA and its ligands regulates humoral immunity as well as the growth and differentiation of B cells from different aspects to maintain a stable and balanced environment in the human body.

The expression of BCMA is restricted to B cell lines. It is expressed on plasma blasts, plasma cells and a portion of mature B cells, and increased at the differentiation of terminal B cells. While in most B cells, such as naive B cells, memory B cells and B cell germinal centers and other organs, BCMA is not expressed. It has been reported that the expression of BCMA is important for long-lived, fixed plasma cells in the bone marrow. Therefore, plasma cells in the bone marrow are reduced in BCMA-deficient mice, but plasma cell level in the spleen is not affected. Mature B cells can normally differentiate into plasma cells in BCMA knockout mice. The BCMA knockout mice looked normal and seemed healthy, and the number of B cells was normal, but the plasma cells could not survive for a long time.

BCMA is also highly expressed in malignant plasma cells, such as multiple myeloma and plasma cell leukemia. BCMA is also detected in HRS cells of patients with Hodgkin's lymphoma. In America, malignant tumors of blood system account for about 10% of all malignant tumors, and myeloma accounts for 15% of all malignant hematological tumors. According to the literature, the expression of BCMA is associated with progression of multiple myeloma disease. The BCMA gene is highly expressed in myeloma samples, but is low expressed in chronic lymphocytic leukemia, acute lymphocytic leukemia, and acute T-cell lymphocytic leukemia. B cell lymphomas were significantly increased in a mouse model overexpressing BCMA ligands BAFF and APRIL. Ligands that bind to BCMA have been shown to regulate the growth and survival of multiple myeloma cells expressing BCMA. The combination of BCMA with BAFF and APRIL can make malignant plasma cells survive. Therefore, loss of tumor cells expressing BCMA and distribution of the interaction between BCMA ligand and receptor can improve outcome in the treatment of multiple myeloma or other BCMA positive B cell lines malignant lymphoma.

Multiple myeloma, also known as plasmacytoma or Keller's disease, is a malignant tumor of the refractory B cell line, characterized by abnormal proliferation of plasma cells. Plasma cells are a type of leukocyte that is responsible for production of antibodies. According to data released by the National Cancer Institute in 2017, myeloma accounts for 1.8% of all tumor cases, with a mortality rate of 2.1%. The statistical results of 2010-2014 show that the incidence rate is about 6.6 in 100,000 per year and the mortality rate is about 50%. Multiple myeloma is a middle-aged disease. The median age of onset in Europe and the United States is 68 years old. There are more males than females. The peak age of onset in China is 55-65 years old, and the ratio of male to female is 2.35:1. There is no confirmed epidemiological data on multiple myeloma in China. It is generally estimated that the incidence rate is similar to that in surrounding southeast Asia and Japan, about one in 100,000. Traditional treatments for multiple myeloma include chemotherapy and hematopoietic stem cell transplantation, but these methods have a high recurrence rate. Bortezomib (PS-341) is first proteasome inhibitor, which is approved by the FDA in 2003 for the treatment of relapsed refractory multiple myeloma, either alone or in combination with existing medications. The results were gratifying. The drug was also marketed in China in 2005 and has become one of the options for the treatment of multiple myeloma with thalidomide and dexamethasone. The treatment of multiple myeloma is usually combined. However, if multiple drugs are used at the same time, there are also negative effects of costly and cumulative side effects. There is still a clinical need to develop new methods for the treatment of multiple myeloma.

Recently, immunotherapy, especially adoptive T-cell therapy, has shown strong efficacy and bright prospects in clinical trials for the treatment of malignant tumors of the blood system. T cells can be genetically modified to express a chimeric antigen receptor (CAR), which includes an antigen recognition portion and a T cell activation region. Using the antigen binding properties of monoclonal antibodies, CAR can redirect the specificity and reactivity of T cell and target in a non-MHC restricted manner. This non-MHC restricted antigen recognition allows CAR-expressing T cells to recognize antigen without antigen processing, thus avoiding a major mechanism of tumor escape. In addition, CAR does not produce dimers with alpha chain and beta chain of the endogenous TCR.

At present, two chimeric antigen receptor T cell therapy (CAR-T) products targeting CD19 have been approved for the treatment of acute lymphoblastic leukemia in children and young adult patients and adult second-line or multi-line system therapy of recurrent or refractory large B-cell lymphoma. However, CD19 is rarely expressed in malignant plasma cells of multiple myeloma. There is an urgent need to develop a CAR-T product that targets BCMA for the treatment of multiple myeloma.

### SUMMARY

It is an object of the present disclosure to provide chimeric antigen receptors targeting BCMA as well as preparation methods and applications thereof.

The present disclosure provides for a chimeric antigen receptor (CAR). The CAR may comprise: an anti-BCMA antigen-binding region which comprises a light chain variable region (V_{L}) and a heavy chain variable region (V_{H}).

V_{L} may comprise three complementarity determining regions (CDRs), LCDR1, LCDR2 and LCDR3; V_{H} may comprise three CDRs, HCDR1, HCDR2 and HCDR3.

In certain embodiments, LCDR1, LCDR2 and LCDR3 may have amino acid sequences about 80% to about 100% identical to the amino acid sequences set forth in SEQ ID NO: 17, SEQ ID NO: 19, SEQ ID NO: 21, respectively. HCDR1, HCDR2 and HCDR3 may have amino acid sequences about 80% to about 100% identical to the amino acid sequences set forth in SEQ ID NO: 24, SEQ ID NO: 26, SEQ ID NO: 28, respectively.

In certain embodiments, LCDR1, LCDR2 and LCDR3 may have amino acid sequences about 80% to about 100% identical to the amino acid sequences set forth in SEQ ID NO: 31, SEQ ID NO: 33, SEQ ID NO: 35, respectively. HCDR1, HCDR2 and HCDR3 may have amino acid sequences about 80% to about 100% identical to the amino acid sequences set forth in SEQ ID NO: 38, SEQ ID NO: 40, SEQ ID NO: 42, respectively.

In certain embodiments, LCDR1, LCDR2 and LCDR3 may have amino acid sequences about 80% to about 100% identical to the amino acid sequences set forth in SEQ ID NO: 45, SEQ ID NO: 47, SEQ ID NO: 49, respectively. HCDR1, HCDR2 and HCDR3 may have amino acid sequences about 80% to about 100% identical to the amino acid sequences set forth in SEQ ID NO: 52, SEQ ID NO: 54, SEQ ID NO: 56, respectively.

V_{L} and V_{H} of the CAR may have amino acid sequences about 80% to about 100% identical to amino acid sequences set forth in (a) SEQ ID NO: 1 and SEQ ID NO: 2, respectively; (a) SEQ ID NO: 3 and SEQ ID NO: 4, respectively; or (a) SEQ ID NO: 5 and SEQ ID NO: 6, respectively.

In certain embodiments, V_{L} is located at the N-terminus of V_{H}.

The anti-BCMA antigen-binding region may be a single-chain variable fragment (scFv) that specifically binds BCMA.

The CAR may further comprise one or more of the following: (a) a signal peptide, (b) a hinge region, (c) a transmembrane domain, (d) a co-stimulatory region, and (e) a cytoplasmic signaling domain.

The co-stimulatory region may comprise a co-stimulatory region of (or may be derived from) 4-1BB (CD137), CD28, OX40, CD2, CD7, CD27, CD30, CD40, CD70, CD134, PD1, Dap10, CDS, ICAM-1, LFA-1 (CD11a/CD18), ICOS (CD278), NKG2D, GITR, TLR2, or combinations thereof.

The cytoplasmic signaling domain may comprise a cytoplasmic signaling domain of (or may be derived from) CD3ζ.

The hinge region may comprise a hinge region of (or may be derived from) CD8, CD28, CD137, Ig4, or combinations thereof.

The transmembrane domain may comprise a transmembrane domain of (or may be derived from) CD8, CD28, CD3, CD45, CD4, CD5, CD9, CD16, CD22, CD33, CD37, CD64, CD80, CD86, CD134, CD137, CD154, or combinations thereof.

The present disclosure provides for an immune cell expressing the present CAR. The immune cell may be a T cell or a natural killer (NK) cell. The immune cell may be allogeneic or autologous.

Also encompassed by the present disclosure is a nucleic acid encoding the present CAR.

The present disclosure further provides for a vector comprising the present nucleic acid.

The present disclosure provides for a method of treating cancer. The method may comprise administering the immune cell to a subject in need thereof.

The cancer may be a hematologic cancer. The cancer may be a plasma-cell malignancy. The cancer may be a BCMA-positive malignancy. The cancer may be multiple myeloma (MM), or plasma cell leukemia.

The immune cell may be administered by infusion, injection, transfusion, implantation, and/or transplantation.

The immune cell may be administered intravenously, subcutaneously, intradermally, intranodally, intratumorally, intramedullary, intramuscularly, or intraperitoneally.

The immune cell may be administered via intravenous infusion.

The subject may be a human.

The present disclosure provides for a method for treating cancer. The method may comprise administering the present immune cell to a subject in need of.

The chimeric antigen receptor (CAR) may generate an area under the curve (AUC) ranging from about 5.0e+05 copies/vg genomic DNA (copies/gDNA) to about 1.3e+07 copies/gDNA, from about 5.0e+06 copies/vg genomic DNA (copies/gDNA) to about 1.0e+07 copies/gDNA, from about 5.0e+06 copies/vg genomic DNA (copies/gDNA) to about 1.3e+07 copies/gDNA, or from about 7.0e+06 copies/vg genomic DNA (copies/gDNA) to about 1.0e+07 copies/gDNA, in the blood of the subject in about 28 days after administration.

The chimeric antigen receptor (CAR) may generate a maximum plasma concentration (Cₘₐₓ) ranging from about 5×10⁴ copies/vg genomic DNA (copies/gDNA) to about 1.3×10⁶ copies/gDNA, from about 5×10⁵ copies/vg genomic DNA (copies/gDNA) to about 1.3×10⁶ copies/gDNA, or from about 7.5×10⁵ copies/vg genomic DNA (copies/gDNA) to about 1×10⁶ copies/gDNA, in the blood of the subject.

The CAR may have a Tₘₐₓ ranging from about 12 days to about 25 days, from about 14 days to about 20 days, or from about 6 days to about 22 days.

In certain embodiments, the anti-BCMA antigen-binding region includes a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 1, SEQ ID NO: 3, or SEQ ID NO: 5.

In certain embodiments, the anti-BCMA antigen-binding region includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 2, SEQ ID NO: 4, or SEQ ID NO: 6.

A light chain variable region of the anti-BCMA antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a light chain variable region of the BCMA-20 antibody (CDR1, CDR2 and CDR3 as set forth in position 24-34, position 50-56, position 89-97 of SEQ ID NO: 1, respectively), or the CDRs of a light chain variable region of the BCMA-CA8 antibody (CDR1, CDR2 and CDR3 as set forth in position 24-34, position 50-56, position 89-97 of SEQ ID NO: 3, respectively), or the CDRs of a light chain variable region of the BCMA-MO6 antibody (CDR1, CDR2 and CDR3 as set forth in position 24-34, position 50-56, position 89-97 of SEQ ID NO: 5, respectively).

A heavy chain variable region of the anti-BCMA antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a heavy chain variable region of the BCMA-20 antibody (CDR1, CDR2 and CDR3 as set forth in position 31-35, position 50-66, position 99-110 of SEQ ID NO: 2, respectively), or the CDRs of a heavy chain variable region of the BCMA-CA8 antibody (CDR1, CDR2 and CDR3 as set forth in position 31-35, position 50-66, position 99-110 of SEQ ID NO: 4, respectively), or the CDRs of a heavy chain variable region of the BCMA-MO6 antibody (CDR1, CDR2 and CDR3 as set forth in position 31-35, position 50-66, position 99-110 of SEQ ID NO: 6, respectively).

A light chain variable region of the anti-BCMA antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a light chain variable region of the BCMA-20 antibody (CDR1, CDR2 and CDR3 as set forth in SEQ ID NO: 17, SEQ ID NO: 19, SEQ ID NO: 21, respectively), or the CDRs of a light chain variable region of the BCMA-CA8 antibody (CDR1, CDR2 and CDR3 as set forth in SEQ ID NO: 31, SEQ ID NO: 33, SEQ ID NO: 35, respectively), or the CDRs of a light chain variable region of the BCMA-MO6 antibody (CDR1, CDR2 and CDR3 as set forth in SEQ ID NO: 45, SEQ ID NO: 47, SEQ ID NO: 49, respectively).

A heavy chain variable region of the anti-BCMA antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a heavy chain variable region of the BCMA-20 antibody (CDR1, CDR2 and CDR3 as set forth in SEQ ID NO: 24, SEQ ID NO: 26, SEQ ID NO: 28, respectively), or the CDRs of a heavy chain variable region of the BCMA-CA8 antibody (CDR1, CDR2 and CDR3 as set forth in SEQ ID NO: 38, SEQ ID NO: 40, SEQ ID NO: 42, respectively), or the CDRs of a heavy chain variable region of the BCMA-MO6 antibody (CDR1, CDR2 and CDR3 as set forth in SEQ ID NO: 52, SEQ ID NO: 54, SEQ ID NO: 56, respectively).

In certain embodiments, a light chain variable region of the anti-BCMA antigen-binding region includes three CDRs that are identical (e.g., 80%-100% identical) to CDRs of a heavy chain variable region of the BCMA-20 antibody (CDR1, CDR2 and CDR3 as set forth in position 24-34, position 50-56, position 89-97 of SEQ ID NO: 1), and a heavy chain variable region of the anti-BCMA antigen-binding region includes three CDRs that are identical (e.g., 80%-100% identical) to CDRs of a heavy chain variable region of the BCMA-20 antibody (CDR1, CDR2 and CDR3 as set forth in position 31-35, position 50-66, position 99-110 of SEQ ID NO: 2, respectively).

In certain embodiments, a light chain variable region of the anti-BCMA antigen-binding region includes three CDRs that are identical (e.g., 80%-100% identical) to the CDRs of a light chain variable region of the BCMA-20 antibody (CDR1, CDR2 and CDR3 as set forth in SEQ ID NO: 17, SEQ ID NO: 19, SEQ ID NO: 21, respectively), and a heavy chain variable region of the anti-BCMA antigen-binding region includes three CDRs that are identical (e.g., 80%-100% identical) to the CDRs of a heavy chain variable region of the BCMA-20 antibody (CDR1, CDR2 and CDR3 as set forth in SEQ ID NO: 24, SEQ ID NO: 26, SEQ ID NO: 28, respectively).

In certain embodiments, a light chain variable region of the anti-BCMA antigen-binding region includes three CDRs that are identical (e.g., 80%-100% identical) to CDRs of a light chain variable region of the BCMA-CA8 antibody (CDR1, CDR2 and CDR3 as set forth in position 24-34, position 50-56, position 89-97 of SEQ ID NO: 3, respectively), and a heavy chain variable region of the anti-BCMA antigen-binding region includes three CDRs that are identical (e.g., 80%-100% identical) to CDRs of a heavy chain variable region of the BCMA-CA8 antibody (CDR1, CDR2 and CDR3 as set forth in position 31-35, position 50-66, position 99-110 of SEQ ID NO: 4, respectively).

In certain embodiments, a light chain variable region of the anti-BCMA antigen-binding region includes three CDRs that are identical (e.g., 80%-100% identical) to the CDRs of a light chain variable region of the BCMA-CA8 antibody (CDR1, CDR2 and CDR3 as set forth in SEQ ID NO: 31, SEQ ID NO: 33, SEQ ID NO: 35, respectively), and a heavy chain variable region of the anti-BCMA antigen-binding region includes three CDRs that are identical (e.g., 80%-100% identical) to the CDRs of a heavy chain variable region of the BCMA-CA8 antibody (CDR1, CDR2 and CDR3 as set forth in SEQ ID NO: 38, SEQ ID NO: 40, SEQ ID NO: 42, respectively).

In certain embodiments, a light chain variable region of the anti-BCMA antigen-binding region includes three CDRs that are identical to CDRs of a light chain variable region of the BCMA-MO6 antibody (CDR1, CDR2 and CDR3 as set forth in position 24-34, position 50-56, position 89-97 of SEQ ID NO: 5, respectively), and a heavy chain variable region of the anti-BCMA antigen-binding region includes three CDRs that are identical to CDRs of a heavy chain variable region of the BCMA-MO6 antibody (CDR1, CDR2 and CDR3 as set forth in position 31-position 50-66, position 99-110 of SEQ ID NO: 6, respectively).

In certain embodiments, a light chain variable region of the anti-BCMA antigen-binding region includes three CDRs that are identical (e.g., 80%-100% identical) to the CDRs of a light chain variable region of the BCMA-MO6 antibody (CDR1, CDR2 and CDR3 as set forth in SEQ ID NO: 45, SEQ ID NO: 47, SEQ ID NO: 49, respectively), and a heavy chain variable region of the anti-BCMA antigen-binding region includes three CDRs that are identical (e.g., 80%-100% identical) to the CDRs of a heavy chain variable region of the BCMA-MO6 antibody (CDR1, CDR2 and CDR3 as set forth in SEQ ID NO: 52, SEQ ID NO: 54, SEQ ID NO: 56, respectively).

In certain embodiments, the CAR may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 59, SEQ ID NO: 61, or SEQ ID NO: 63.

In certain embodiments, the nucleic acid encoding the CAR may comprise a nucleic acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 58, SEQ ID NO: 60, or SEQ ID NO: 62.

In certain embodiments, the CAR may generate an area under the curve (AUC) ranging from about 0.5e+06 copies/μg genomic DNA (copies/gDNA) to about 2e+07 copies/gDNA, from about 5.0e+05 copies/vg genomic DNA (copies/gDNA) to about 1.3e+07 copies/gDNA, from about 5.0e+05 copies/vg genomic DNA (copies/gDNA) to about 2e+07 copies/gDNA, from about copies/vg genomic DNA (copies/gDNA) to about 1.5e+07 copies/gDNA, from about copies/vg genomic DNA (copies/gDNA) to about 1.0e+07 copies/gDNA, from about 5.0e+06 copies/vg genomic DNA (copies/gDNA) to about 1.3e+07 copies/gDNA, from about 7.0e+06 copies/vg genomic DNA (copies/gDNA) to about 1.0e+07 copies/gDNA, from about 8.0e+06 copies/vg genomic DNA (copies/gDNA) to about 1.0e+07 copies/gDNA, from about copies/vg genomic DNA (copies/gDNA) to about 4e+06 copies/gDNA, from about copies/vg genomic DNA (copies/gDNA) to about 3.5e+06 copies/gDNA, from about 1e+06 copies/vg genomic DNA (copies/gDNA) to about 3.5e+06 copies/gDNA, from about 1.2e+06 copies/vg genomic DNA (copies/gDNA) to about 3.2e+06 copies/gDNA, from about copies/vg genomic DNA (copies/gDNA) to about 3.2e+06 copies/gDNA, from about 1.6e+06 copies/vg genomic DNA (copies/gDNA) to about 3.2e+06 copies/gDNA, from about 1e+06 copies/vg genomic DNA (copies/gDNA) to about 2e+06 copies/gDNA, from about 0.6e+06 copies/vg genomic DNA (copies/gDNA) to about 1.8e+06 copies/gDNA, from about 3e+06 copies/vg genomic DNA (copies/gDNA) to about 3.2e+06 copies/gDNA, from about 0.5e+06 copies/vg genomic DNA (copies/gDNA) to about 1.7e+06 copies/gDNA, from about 2e+06 copies/vg genomic DNA (copies/gDNA) to about 3.2e+06 copies/gDNA, from about 1.5e+06 copies/vg genomic DNA (copies/gDNA) to about 2e+06 copies/gDNA, or from about 1e+06 copies/vg genomic DNA (copies/gDNA) to about 3.2e+06 copies/gDNA, in the blood of the subject in about 28 days after administration of the CAR to the subject. The AUC may be a median AUC.

In certain embodiments, the CAR generates a maximum plasma concentration (Cₘₐₓ) ranging from about 5×10⁴ copies/vg genomic DNA (copies/gDNA) to about 1.3×10⁶ copies/gDNA, from about 5×10⁴ copies/vg genomic DNA (copies/gDNA) to about 1.5×10⁶ copies/gDNA, from about 5×10⁵ copies/vg genomic DNA (copies/gDNA) to about 1.3×10⁶ copies/gDNA, from about 7.5×10⁵ copies/vg genomic DNA (copies/gDNA) to about 1×10⁶ copies/gDNA, from about 7×10⁵ copies/vg genomic DNA (copies/gDNA) to about 1×10⁶ copies/gDNA, from about 8×10⁵ copies/vg genomic DNA (copies/gDNA) to about 1×10⁶ copies/gDNA, from about 7.5×10⁵ copies/vg genomic DNA (copies/gDNA) to about 1.5×10⁶ copies/gDNA, from about 7×10⁵ copies/vg genomic DNA (copies/gDNA) to about 1.5×10⁶ copies/gDNA, from about 8×10⁵ copies/vg genomic DNA (copies/gDNA) to about 1.5×10⁶ copies/gDNA, from about 0.8e+05 copies/vg genomic DNA (copies/gDNA) to about 3.5e+05 copies/gDNA, from about 1e+05 copies/vg genomic DNA (copies/gDNA) to about 3.5e+05 copies/gDNA, from about 1e+05 copies/vg genomic DNA (copies/gDNA) to about 1.6e+05 copies/gDNA, from about 1e+05 copies/vg genomic DNA (copies/gDNA) to about 3.3e+05 copies/gDNA, from about 0.8e+05 copies/vg genomic DNA (copies/gDNA) to about 1.5e+05 copies/gDNA, from about 0.8e+05 copies/vg genomic DNA (copies/gDNA) to about 2e+05 copies/gDNA, from about 1e+05 copies/vg genomic DNA (copies/gDNA) to about 2e+05 copies/gDNA, from about 2e+05 copies/vg genomic DNA (copies/gDNA) to about 3e+05 copies/gDNA, from about 2e+05 copies/vg genomic DNA (copies/gDNA) to about 3.5e+05 copies/gDNA, from about 2e+05 copies/vg genomic DNA (copies/gDNA) to about 2.5e+05 copies/gDNA, or from about 1e+05 copies/vg genomic DNA (copies/gDNA) to about 3e+05 copies/gDNA, in the blood of the subject after administration of the CAR to the subject. The Cₘₐₓ may be a median Cₘₐₓ.

In certain embodiments, the CAR has a Tₘₐₓ (the time it takes the CAR to reach Cₘₐₓ) ranging from about 12 days to about 25 days, from about 14 days to about 20 days, from about 6 days to about 22 days, from about 3 days to about 20 days, from about 4 days to about 18 days, from about 5 days to about 17 days, from about 6 days to about 16 days, from about 7 days to about 15 days, from about 9 days to about 15 days, from about 10 days to about 15 days, from about 10 days to about 14 days, from about 8 days to about 12 days, from about 6 days to about 14 days, from about 12 days to about 14 days, from about 8 days to about 11 days, from about 8 days to about 15 days, or from about 10 days to about 14 days. The Tₘₐₓ may be a median Tₘₐₓ.

In certain embodiments, the CAR has a Tₗₐₛₜ (the time corresponding to the last quantifiable CAR level) ranging from about 30 days to about 200 days, from about 50 days to about 150 days, from about 50 days to about 100 days, from about 60 days to about 80 days, from about 60 days to about 150 days, from about 80 days to about 150 days, from about 50 days to about 200 days, from about 50 days to about 60 days, from about 50 days to about 80 days, from about 50 days to about 100 days, from about 60 days to about 100 days, from about 80 days to about 100 days, from about 60 days to about 200 days, from about 80 days to about 200 days, from about 50 days to about 140 days, from about 60 days to about 140 days, or from about 80 days to about 140 days. The Tₗₐₛₜ may be a median Tₗₐₛₜ.

Specifically, it is an object of the present disclosure to provide a sequence of BCMA targeted chimeric antigen receptor as well as preparation method and activity identification of a modified T cell (CART-BCMA) thereof.

The present disclosure provides a chimeric antigen receptor structure for use in the treatment of BCMA positive B cell lymphoma.

In a first aspect, it provides a chimeric antigen receptor (CAR) (sequence), and its antigen binding domain is an antibody single chain variable region sequence that targets extracellular region of BCMA.

In another embodiment, the antigen binding domain is an antibody single chain variable region sequence that targets amino acid residues at positions 24 to 41 of the BCMA sequence.

In another embodiment, the NCBI accession number of the BCMA sequence is AY684975.1.

In another embodiment, the structure of the antigen binding domain is shown in formula I as below:

V_{L}-V_{H} (I)
wherein V_{H} is an antibody heavy chain variable region; V_{L} is an antibody light chain variable region; and “-” is a linker peptide or a peptide bond;
and, the amino acid sequence of V_{L} is as shown in SEQ ID NO: 1, and the amino acid sequence of V_{H} is as shown in SEQ ID NO: 2;
or, the amino acid sequence of V_{L} is as shown in SEQ ID NO: 3, and the amino acid sequence of V_{H} is as shown in SEQ ID NO: 4;
or, the amino acid sequence of V_{L} is as shown in SEQ ID NO: 5, and the amino acid sequence of V_{H} is as shown in SEQ ID NO: 6.

In another embodiment, the amino acid sequence of the linker peptide is as shown in SEQ ID NO: 10 or SEQ ID NO: 11.

In another embodiment, the antibody single chain variable region comprises a human, mouse, human-mouse chimeric antibody single chain variable region.

In another embodiment, the structure of the chimeric antigen receptor is shown in formula II as below:

S-V_{L}-V_{H}-H-TM-C-CD3ζ (II)
wherein,
S is an optional signal peptide;
H is a hinge region;
TM is a transmembrane domain;
C is a co-stimulatory signaling molecule;
CD3ζ is a cytoplasmic signaling sequence derived from CD3ζ;
V_{H} and V_{L} are as described above.

In another embodiment, the S is a signal peptide of a protein selected from the group consisting of CD8, CD28, GM-CSF, CD4, CD137, or a combination thereof.

In another embodiment, the S is a signal peptide derived from CD8.

In another embodiment, the amino acid sequence of S is as shown in SEQ ID NO: 9.

In another embodiment, the H is a hinge region of a protein selected from the group consisting of CD8, CD28, CD137, or a combination thereof.

In another embodiment, the H is a hinge region derived from CD8.

In another embodiment, the amino acid sequence of H is as shown in SEQ ID NO: 12.

In another embodiment, the TM is a transmembrane region of a protein selected from the group consisting of CD28, CD3, CD45, CD4, CD5, CD8, CD9, CD16, CD22, CD33, CD37, CD64, CD80, CD86, CD134, CD137, CD154, or a combination thereof.

In another embodiment, the TM is a transmembrane region derived from CD8.

In another embodiment, the sequence of TM is as shown in SEQ ID NO: 13.

In another embodiment, the C is a co-stimulatory signaling molecule of a protein selected from the group consisting of OX40, CD2, CD7, CD27, CD28, CD30, CD40, CD70, CD134, 4-1BB (CD137), PD1, Dap10, CDS, ICAM-1, LFA-1 (CD11a/CD18), ICOS (CD278), NKG2D, GITR, TLR2, or a combination thereof.

In another embodiment, C is a co-stimulatory signaling molecule derived from 4-1BB.

In another embodiment, the amino acid sequence of C is as shown in SEQ ID NO: 14.

In another embodiment, the amino acid sequence of CD3ζ is as shown in SEQ ID NO: 15.

In a second aspect, it provides a nucleic acid molecule, encoding the chimeric antigen receptor (CAR) of the first aspect of.

In another embodiment, the nucleic acid molecule is isolated.

In a third aspect, it provides a vector, comprising the nucleic acid molecule of the second aspect.

In another embodiment, the vector is selected from the group consisting of DNA, RNA, plasmid, lentiviral vector, adenoviral vector, retroviral vector, transposon, or a combination thereof.

In another embodiment, the vector is a lentiviral vector.

In a fourth aspect, it provides a host cell, comprising the vector of the third aspect or having the exogenous nucleic acid molecule of the second aspect integrated into the chromosome or expressing the CAR of the first aspect.

In another embodiment, the cell is an isolated cell, and/or the cell is a genetically engineered cell.

In another embodiment, the cell is a mammalian cell.

In another embodiment, the cell is a T cell.

In a fifth aspect, it provides a method for preparing a CAR-T cell expressing the CAR of the first aspect, and the method comprises the steps of: transducing the nucleic acid molecule of the second aspect or the vector of the third aspect into a T cell, thereby obtaining the CAR-T cell.

In a sixth aspect, it provides a preparation, comprising the chimeric antigen receptor of the first aspect, the nucleic acid molecule of the second aspect, the vector of the third aspect, or the cell of the fourth aspect, and a pharmaceutically acceptable carrier, diluent or excipient.

In another embodiment, the preparation is a liquid preparation.

In another embodiment, the dosage form of the preparation is injection.

In another embodiment, the concentration of the CAR-T cells in the preparation is 1×10³-1×10⁸ cells/ml, or 1×10⁴-1×10⁷ cells/ml.

In a seventh aspect, it provides the use of the chimeric antigen receptor of the first aspect, the nucleic acid molecule of the second aspect, the vector of the third aspect, or the cell of the fourth aspect, for the preparation of a medicine or a preparation for preventing and/or treating tumor or cancer.

In another embodiment, the tumor is selected from the group consisting of a hematological tumor, a solid tumor, or a combination thereof.

In one embodiment, the cancer is B cell lymphoma.

In another embodiment, the blood tumor is selected from the group consisting of acute myeloid leukemia (AML), multiple myeloma (MM), chronic lymphocytic leukemia (CLL), acute lymphoblastic leukemia (ALL), diffuse large B cell lymphoma (DLBCL), or a combination thereof.

In another embodiment, the solid tumor is selected from the group consisting of gastric cancer, peritoneal metastasis of gastric cancer, liver cancer, leukemia, renal cancer, lung cancer, small intestine cancer, bone cancer, prostate cancer, colorectal cancer, breast cancer, large intestine cancer, cervical cancer, ovarian cancer, lymphoma, nasopharyngeal carcinoma, adrenal tumor, bladder tumor, non-small cell lung cancer (NSCLC), glioma, endometrial cancer, or a combination thereof.

In another embodiment, the tumor is a BCMA positive tumor, such as a BCMA positive B cell lymphoma, multiple myeloma, or plasma cell leukemia.

In an eighth aspect, it provides a kit for the preparation of the cell of the fourth aspect, the kit comprises a container, and the nucleic acid molecule of the second aspect or the vector of the third aspect is located in the container.

In a ninth aspect, it provides a use of the cell of the fourth aspect, or the preparation of the sixth aspect for the prevention and/or treatment of cancer or tumor.

In a tenth aspect, it provides a method of treating a disease comprising administering an appropriate amount of the cell of the fourth aspect, or the preparation of the sixth aspect, to a subject in need of treatment.

In another embodiment, the disease is cancer or tumor.

It is to be understood that the various technical features of the present disclosure mentioned above and the various technical features specifically described hereinafter (as in the Examples) may be combined with each other within the scope of the present disclosure to constitute a new or preferred technical solution, which needs not be described one by one, due to space limitations.

### BRIEF DESCRIPTION OF THE DRAWINGS

FIGS. 1A, 1B, and 1C show screening result of CART-BCMA comparative examples. FIG. 1A shows detection of transfection efficiency of engineered T cell with chimeric antigen receptors targeting BCMA. The expression level of the CAR gene-encoded protein on the surface of the T cell membrane in CAR-BCMA cells cultured on day 6 was identified by Fc fragment staining method of recombinant human BCMA protein. 1*10⁵ of CART-BCMA cells cultured on day 10 were cultured respectively with BCMA-positive K562-BCMA-B9 tumor cell line, MM.1S and RPMI8226 tumor cell lines naturally expressing BCMA, and BCMA-negative K562 tumor cell line, or without tumor cells, in 200 μl GT-551 medium for 18 h at a ratio of 1:1. Then the expression level of CD137 on the surface of T cell membrane (FIG. 1B) and the secretion level of IFNγ in the culture supernatant was detected respectively (FIG. 1C).

FIG. 2 shows structure of chimeric antigen receptor targeting BCMA. The structure of CAR includes a leader sequence, an antigen recognition sequence, a linker region, a transmembrane region, a co-stimulatory factor signal region, and a CD3ζ signaling region.

FIGS. 3A, 3B, and 3C show detection of transfection efficiency of engineered T cell with chimeric antigen receptors targeting BCMA. The expression levels of the CAR gene-encoded protein on the surface of the T cell membrane in CAR-BCMA cells cultured on day 7 (FIG. 3A) day 21 (FIG. 3B) and day 29 (FIG. 3C) were identified by Fc fragment staining method of recombinant human BCMA protein.

FIGS. 4A and 4B show the expression level of CD137 on the surface of T cell membrane (FIG. 4A) and the secretion level of IFNγ in the culture supernatant (FIG. 4B). Specifically, 1×10⁵ of CART-BCMA cells cultured on day 7 were cultured respectively with BCMA-positive K562-BCMA-E7 tumor cell line and BCMA-negative K562 tumor cell line, or without tumor cells, in 200 μl GT-551 medium for 18 h at a ratio of 1:1. Then the expression level of CD137 on the surface of T cell membrane and the secretion level of IFNγ in the culture supernatant was detected respectively.

FIG. 5 shows detection of advanced apoptosis level of tumor cells induced by CART-BCMAs. Specifically, 1×10⁴ CFSE-labeled BCMA-negative (NH929) or BCMA-positive (NH929-BCMA) tumor cell lines were co-cultured respectively with corresponding T cells in 100 μl of GT-551 medium for 4 h at a ratio as shown in the figure. The proportion of PI-positive cells in CFSE-positive cells was analyzed by flow cytometry after staining with 100 μl of 25% PI dye for 15 min. The figure shows the statistical analysis of PI positive cells in the corresponding co-culture samples.

FIGS. 6A and 6B show detection of advanced apoptosis level of tumor cells induced by CART-BCMAs. FIG. 6A shows ratio of CART positive cells in the analyzed samples, wherein NT and BCMA-MO6 were calculated at 60%. 1×10⁴ CFSE-labeled BCMA-negative (NH929) or BCMA-positive (NH929-BCMA, MM.1S) tumor cell lines were co-cultured respectively with corresponding T cells in 100 μl of GT-551 medium for 4 h at a ratio as shown in the figure. The proportion of PI-positive cells in CFSE-positive cells was analyzed by flow cytometry after staining with 100 μl of 25% PI dye for 15 min. FIG. 6B shows the statistical analysis of PI positive cells in the corresponding co-culture samples.

FIGS. 7A and 7B show the inhibitory effect of CART-BCMAs on the proliferation in vivo of myeloma cell line RPMI-8226 in B-NDG mice. RPMI-8226 cells in logarithmic growth phase were collected, and 4.0×10⁶ tumor cells were inoculated subcutaneously in the right back of mice. When the tumor volume reached about 120 mm³, the animals were randomly divided into 4 groups according to tumor volume. Then solvent control, 7.5×10⁶ NT (T cells only) and 7.5×10⁶ CART-BCMAs cells were injected through tail vein respectively. FIG. 7A shows that single injection of CART-BCMA-1 and CART-BCMA-20 through tail vein can effectively inhibit the growth of human myeloma RPMI-8226 cells. FIG. 7B shows that CART-BCMA-1 and CART-BCMA-20 can significantly prolong the survival of tumor-bearing mice with human myeloma RPMI-8226 cells.

FIG. 8 shows results the expression of BCMA, CR2, CXADR, DDR2, and MAG in 293T cells after transfection of relevant plasmids by flow cytometry.

FIGS. 9A, 9B, and 9C show the cytokine detection results.

FIG. 10 shows the killing ability of each cell to the target cell. Triangle: BCMA-20 (C-CAR088); square: positive control; circle: NT control (non-transfected (NT) T cells).

FIG. 11 shows the effect of soluble BCMA on cell killing activity.

FIG. 12 shows the survival rate of the mice in each group.

FIG. 13 shows the experimental process of phase I clinical study.

FIG. 14 shows the clinical response of phase I clinical study.

FIG. 15 shows the treatment condition of the patient of ID Z0203-00801C008.

FIGS. 16A and 16B show the treatment condition of patient of ID Z0203-00701C001.

FIG. 17 shows the expansion of C-CAR088 and the decrease of M-protein/sFLC levels in the blood.

FIGS. 18A-18C show the results of evaluating the binding specificity of the scFv of C-CAR088. FIG. 18A shows the experimental scheme. FIG. 18B shows the structure of the anti-BCMA CAR and scFv rabFc. FIG. 18C shows the experimental results.

FIGS. 19A-19B show tissue cross reactivity IHC GLP study and validation. HAdCC, human primary adrenal cortical cells. HPTEC, human primary thyroid epithelial cells. A549 BCMA OE, a stable strain overexpressing BCMA.

FIG. 20 shows an example of the CAR (C-CAR088) manufacture process. The process includes the usage of serum free media, a closed, modular integrated, semi-automated system, and digital monitoring. Stars indicate improved processes. Median vein to vein time is about 17 days (range: 13 to 83 days). Median manufacturing time is about 7 days (range: 5 to 10 days).

FIG. 21 shows C-CAR088 phase I clinical study design (in treating relapsed or refractory multiple myeloma). Phase I, open-label, dose escalation and expansion studies were conducted at four medical centers. C-CAR088 is an embodiment of the present CAR which is based on the BCMA-20 antibody.

FIG. 22 shows the C-CAR088 clinical response, including SD, MR, PR, CR, VGPR, MRD, and PD. SD: stable disease; PR: partial response; CR: complete response; PD: progressive disease; MR: minimal response; VGPR: very good partial response; MRD: minimal residual disease.

FIG. 23 shows the C-CAR088 clinical response, including CR/sCR, VGPR and PR.

FIG. 24 shows the Kaplan Meyer estimation of progression-free survival (PFS) for mid and high dose group.

FIG. 25A shows the expansion of C-CAR088 in the blood of the patients after CAR administration. FIG. 25B shows the expansion of C-CAR088 in the blood of the patients up to the most recent visit. FIG. 25C shows Cₘₐₓ levels in the blood of the patients after CAR administration. FIG. 25D shows AUC levels in the blood of the patients after CAR administration. FIG. 25E shows Tₘₐₓ levels in the blood of the patients after CAR administration. FIG. 25F shows Tₗₐₛₜ levels in the blood of the patients after CAR administration.

### DETAILED DESCRIPTION

The present disclosure provides for chimeric antigen receptors (CARs) targeting BCMA. In certain embodiments, the CARs are based on four monoclonal antibodies: BCMA-1, BCMA-20, BCMA-CA8, and BCMA-MO6. The present disclosure also provides for the analysis and identification of the expression levels of the CARs in primary T cells, in vitro activation ability and tumor cell killing efficacy of these chimeric antigen receptors. Studies have shown that the chimeric antigen receptors of the present disclosure target BCMA positive cells and can be used to treat BCMA positive B cell lymphoma, multiple myeloma, plasma cell leukemia or other diseases.

Specifically, the present disclosure identifies the correlation between the expression time and the expression intensity of different CAR structures on the surface of the cell membrane after virus infection, and further identifies the difference in expression of different CAR structural proteins. This finding suggests that different CAR structures exist a difference in the expression level of CAR protein on the membrane surface and the persistence of CART in vivo activity under same infection condition. After extensive screening, the CAR structure of the present disclosure was obtained. The results show that the protein encoded by the CAR structure of the present disclosure can be fully expressed and membrane-localized.

In the present disclosure, the preparation process of CAR-modified T cell targeting BCMA antigen is improved. In one embodiment, GT-551 serum-free medium supplemented with 1% human albumin is used to culture lymphocytes in vitro.

Term

The term “about” may refer to a value or composition within an acceptable error range for a particular value or composition as determined by those skilled in the art, which will depend in part on how the value or composition is measured or determined. The term “about” in reference to a numeric value may refer to ±10% of the stated numeric value. In other words, the numeric value can be in a range of 90% of the stated value to 110% of the stated value.

The term “administering” refers to the physical introduction of a product of the disclosure into a subject using any one of various methods and delivery systems known to those skilled in the art, including, but not limited to, intravenous, intramuscular, subcutaneous, intraperitoneal, spinal or other parenteral administration, such as by injection or infusion.

The term “antibody” (Ab) may include, but is not limited to, an immunoglobulin that specifically binds an antigen and contains at least two heavy (H) chains and two light (L) chains linked by disulfide bonds, or an antigen binding parts thereof. Each H chain contains a heavy chain variable region (abbreviated herein as V_{H}) and a heavy chain constant region. The heavy chain constant region contains three constant domains, CH1, CH2, and CH3. Each light chain contains a light chain variable region (abbreviated herein as V_{L}) and a light chain constant region. The light chain constant region contains a constant domain CL. The V_{H} and V_{L} regions can be further subdivided into hypervariable regions called complementarity determining regions (CDR), which are interspersed within more conservative regions called framework regions (FR). Each V_{H} and V_{L} contains three CDRs and four FRs, which are arranged from amino terminal to carboxy terminal in the following order: FR1, CDR1, FR2, CDR2, FR3, CDR3, FR4. The variable regions of the heavy and light chains contain a binding domain that interacts with an antigen.

Chimeric Antigen Receptors (CARs)

The chimeric antigen receptors (CARs) of the present disclosure may comprise an extracellular domain, a transmembrane domain, and an intracellular domain. The extracellular domain comprises a target-specific binding element (also known as an antigen binding domain). The intracellular domain includes a co-stimulatory signaling region and a chain. The co-stimulatory signaling region refers to a part of the intracellular domain that includes a co-stimulatory molecule. The co-stimulatory molecule is a cell surface molecule required for efficient response of lymphocytes to antigens, rather than an antigen receptor or its ligand.

A linker can be incorporated between the extracellular domain and the transmembrane domain of the CAR, or between the cytoplasmic domain and the transmembrane domain of the CAR. As used herein, the term “linker” generally refers to any oligopeptide or polypeptide that plays a role of linking the transmembrane domain to the extracellular domain or the cytoplasmic domain in a polypeptide chain. The linker may comprise 0-300 amino acids, 2-100 amino acids, or 3-50 amino acids.

In one embodiment, the extracellular domain of the CAR comprises an antigen binding domain targeting BCMA. When the CAR of the present disclosure is expressed in T cell, antigen recognition can be performed based on antigen binding specificity. When the CAR binds to its associated antigen, it affects tumor cell, causing tumor cell to fail to grow, to death or to be affected otherwise, causing the patient's tumor burden to shrink or eliminate. The antigen binding domain may be fused to the intracellular domain from one or more of the co-stimulatory molecules and the ζ chain. The antigen binding domain may be fused with an intracellular domain of a combination of a 4-1BB signaling domain and a CD3ζ signaling domain.

As used herein, the “antigen binding domain” and “single-chain antibody fragment” may refer to a Fab fragment, a Fab′ fragment, a F (ab′) 2 fragment, or a single Fv fragment that has antigen-binding activity. The Fv antibody contains the heavy chain variable region and the light chain variable region of the antibody, but has no constant region. The Fv antibody has the smallest antibody fragment with all antigen-binding sites. Generally, Fv antibodies also include a polypeptide linker between the VH and VL domains, and can form the structure required for antigen binding. The antigen binding domain is usually a scFv (single-chain variable fragment). The size of scFv is typically ⅙ of a complete antibody. The single-chain antibody may be an amino acid chain sequence encoded by a nucleotide chain. In certain embodiments, the scFv may comprise an antibody which specifically recognizes the extracellular region of BCMA, such as amino acid residues at positions 24 to 41 of the BCMA sequence. The antibody may be a single chain antibody.

As for the hinge region and the transmembrane region (transmembrane domain), the CAR can be designed to comprise a transmembrane domain fused to the extracellular domain of the CAR. In one embodiment, a transmembrane domain that is naturally associated with one of the domains in the CAR is used. In some embodiments, transmembrane domains may be selected or modified by amino acid substitutions to avoid binding such domains to the transmembrane domain of the same or different surface membrane proteins, thereby minimizing the interaction with other members of the receptor complexes.

The intracellular domain in the CAR comprises the signaling domain of 4-1BB and the signaling domain of CD3ζ.

In certain embodiments, the CAR structure of the present disclosure comprises a signal peptide, an antigen recognition sequence (antigen-binding domain), a linker region, a transmembrane region, a co-stimulatory factor signal region, and a CD3zeta signaling region (ζ chain portion). The order of connection is as follows:

[CD8S]-[VL-Linker-VH]-[hinge-CD8TM[-]4-1BB]-[CD3zeta]

In certain embodiments, the sequence selected in the present disclosure is as follows:
(1) The signal peptide is a signal peptide sequence derived from CD8:

(SEQ ID NO: 9)
MALPVTALLLPLALLLHAARP

(2) light chain (VL) sequence of single-chain variable region derived from BCMA-1 antibody:

(SEQ ID NO: 7)
DIVLTQSPPSLAMSLGKRATISCRASESVTILGSHLIHW
YQQKPGQPPTLLIQLASNVQTGVPARFSGS GSRTDFTL
TIDPVEEDDVAVYYCLQSRTIPRTFGGGTKLEIK

(3) heavy chain (VH) sequence of single-chain variable region derived from BCMA-1 antibody:

(SEQ ID NO: 8)
QIQLVQSGPELKKPGETVKISCKASGYTFTDYSINWVKR
APGKGLKWMGWINTETREPAYAYDFRGRFAFSLETSAST
AYLQINNLKYEDTATYFCALDYSYAMDYWGQGTSVTVSS

Among them, BCMA-1 is an antibody sequence contained in a published Car-T sequence, and is used as a control in the present application.
(4) light chain (VL) sequence of single-chain variable region derived from BCMA-20 antibody:

(SEQ ID NO: 1)
DIQMTQSPSSLSASVGDRVTITCRASQGISNYLNWYQQK
PGKAPKPLIYYTSNLQSGVPSRFSGSGSGT DYTLTISS
LQPEDFATYYCMGQTISSYTFGQGTKLEI

| | | | Length |
|---|---|---|---|
| | | Residues | (number of |
| VL | | of SEQ | amino acid |
| Region | Sequence | ID NO: 1 | residues) |
| LFR1 | DIQMTQSPSSLSASV | 1-23 | 23 |
| | GDRVTITC | | |
| | (SEQ ID NO: 16) | | |
| CDR-L1 | RASQGISNYLN | 24-34 | 11 |
| | (SEQ ID NO: 17) | | |
| LFR2 | WYQQKPGKAPKPLIY | 35-49 | 15 |
| | (SEQ ID NO: 18) | | |
| CDR-L2 | YTSNLQS | 50-56 | 7 |
| | (SEQ ID NO: 19) | | |
| LFR3 | GVPSRFSGSGSGTDYT | 57-88 | 32 |
| | LTISSLQPEDFATYYC | | |
| | (SEQ ID NO: 20) | | |
| CDR-L3 | MGQTISSYT | 89-97 | 9 |
| | (SEQ ID NO: 21) | | |
| LFR4 | FGQGTKLEI | 98-106 | 9 |
| | (SEQ ID NO: 22) | | |

(5) heavy chain (VH) sequence of single-chain variable region derived from BCMA-20 antibody:

(SEQ ID NO: 2)
EVOLVESGGGLVQPGGSLRLSCAASGFTFSNFDMAWVRQAPG
KGLVWVSSITTGADHAIYADSVKGRFTISRDNAKNTLYLQMN
SLRAEDTAVYYCVRHGYYDGYHLFDYWGQGTLVTVSS

| | | Residues | Length |
|---|---|---|---|
| | | of | (number of |
| VH | | SEQ ID | amino acid |
| Region | Sequence | NO: 2 | residues) |
| HFR1 | EVQLVESGGGLVQPG | 1-30 | 30 |
| | GSLRLSCAASGFTFS | | |
| | (SEQ ID NO: 23) | | |
| CDR- | NEDMA | 31-35 | 5 |
| H1 | (SEQ ID NO: 24) | | |
| HFR2 | WVRQAPGKGLVWVS | 36-49 | 14 |
| | (SEQ ID NO: 25) | | |
| CDR- | SITTGADHAIYADSVKG | 50-66 | 17 |
| H2 | (SEQ ID NO: 26) | | |
| HFR3 | RFTISRDNAKNTLYLQ | 67-98 | 32 |
| | MNSLRAEDTAVYYCVR | | |
| | (SEQ ID NO: 27) | | |
| CDR- | HGYYDGYHLFDY | 99-110 | 12 |
| H3 | (SEQ ID NO: 28) | | |
| HFR4 | WGQGTLVTVSS | 111-121 | 11 |
| | (SEQ ID NO: 29) | | |

(6) light chain (VL) sequence of single-chain variable region derived from BCMA-CA8 antibody:

(SEQ ID NO: 3)
DIQLTQTTSSLSASLGDRVTISCSASTTTSNYLNWYQQKPD
GTVELVIYYTSNLHGGGPSRFSGSGSGTDYSLTIGYLEPED
VATYYCQQYRKLPWTFGGGSKLEIKR

| | | | Length |
|---|---|---|---|
| | | Residues | (number |
| | | of | of amino |
| VL | | SEQ ID | acid |
| Region | Sequence | NO: 3 | residues) |
| LFR1 | DIQLTQTTSSLSAS | 1-23 | 23 |
| | LGDRVTISC | | |
| | (SEQ ID NO: 30) | | |
| CDR-L1 | SASTTTSNYLN | 24-34 | 11 |
| | (SEQ ID NO: 31) | | |
| LFR2 | WYQQKPDGTVELVIY | 35-49 | 15 |
| | (SEQ ID NO: 32) | | |
| CDR-L2 | YTSNLHG | 50-56 | 7 |
| | (SEQ ID NO: 33) | | |
| LFR3 | GGPSRFSGSGSGTD | 57-88 | 32 |
| | YSLTIGYLEPEDV | | |
| | ATYYC | | |
| | (SEQ ID NO: 34) | | |
| CDR-L3 | QQYRKLPWT | 89-97 | 9 |
| | (SEQ ID NO: 35) | | |
| LFR4 | FGGGSKLEIKR | 98-108 | 11 |
| | (SEQ ID NO: 36) | | |

(7) heavy chain (VH) sequence of single-chain variable region derived from BCMA-CA8 antibody:

(SEQ ID NO: 4)
EVQLQQSGAVLARPGASVKMSCKGSGYTFTNYWMHWVKQ
RPGQGLEWIGATYRGHSDTYYNQKFKGKAKLTAVTSTST
AYMELSSLTNEDSAVYYCTRGAIYNGYDVLDNWGQGTLV
TVSS

| | | | Length |
|---|---|---|---|
| | | Residues | (number |
| | | of | of amino |
| VH | | SEQ ID | acid |
| Region | Sequence | NO: 4 | residues) |
| HFR1 | EVQLQQSGAVLARPGA | 1-30 | 30 |
| | SVKMSCKGSGYTFT | | |
| | (SEQ ID NO: 37) | | |
| CDR-H1 | NYWMH | 31-35 | 5 |
| | (SEQ ID NO: 38) | | |
| HFR2 | WVKQRPGQGLEWIG | 36-49 | 14 |
| | (SEQ ID NO: 39) | | |
| CDR-H2 | ATYRGHSDTYYNQKF | 50-66 | 17 |
| | KG | | |
| | (SEQ ID NO: 40) | | |
| HFR3 | KAKLTAVTSTSTA | 67-98 | 32 |
| | YMELSSLTNEDSAV | | |
| | YYCTR | | |
| | (SEQ ID NO: 41) | | |
| CDR-H3 | GAIYNGYDVLDN | 99-110 | 12 |
| | (SEQ ID NO: 42) | | |
| HFR4 | WGQGTLVTVSS | 111-121 | 11 |
| | (SEQ ID NO: 43) | | |

(8) light chain (VL) sequence of single-chain variable region derived from BCMA-MO6 antibody:

(SEQ ID NO: 5)
DIQMTQSPSSLSASVGDRVTITCSASQDISNYLNWYQQKP
GKAPKLLIYYTSNLHSGVPSRFSGSGSGTDFTLTISSLQP
EDFATYYCQQYRKLPWTFGQGTKLEIKR

| | | | Length |
|---|---|---|---|
| | | Residues | (number of |
| V_{L} | | of SEQ | amino acid |
| Region | Sequence | ID NO: 5 | residues) |
| LFR1 | DIQMTQSPSSLSASVGDRVTITC (SEQ | 1-23 | 23 |
| | ID NO: 44) | | |
| CDR-L1 | SASQDISNYLN (SEQ ID NO: 45) | 24-34 | 11 |
| LFR2 | WYQQKPGKAPKLLIY (SEQ ID NO: 46) | 35-49 | 15 |
| CDR-L2 | YTSNLHS (SEQ ID NO: 47) | 50-56 | 7 |
| LFR3 | GVPSRFSGSGSGTDFTLTISSLQPEDF | 57-88 | 32 |
| | ATYYC (SEQ ID NO: 48) | | |
| CDR-L3 | QQYRKLPWT (SEQ ID NO: 49) | 89-97 | 9 |
| LFR4 | FGQGTKLEIKR (SEQ ID NO: 50) | 98-108 | 11 |

(9) heavy chain (VH) sequence of single-chain variable region derived from BCMA-MO6 antibody:

(SEQ ID NO: 6)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS

| | | | Length |
|---|---|---|---|
| | | Residues | (number of |
| V_{H} | | of SEQ | amino acid |
| Region | Sequence | ID NO: 6 | residues) |
| HFR1 | QVQLVQSGAEVKKPGSSVKVSCKASG | 1-30 | 30 |
| | GTFS (SEQ ID NO: 51) | | |
| CDR-H1 | NYWMH (SEQ ID NO: 52) | 31-35 | 5 |
| HFR2 | WVRQAPGQGLEWMG (SEQ ID NO: 53) | 36-49 | 14 |
| CDR-H2 | ATYRGHSDTYYNQKFKG (SEQ ID NO: | 50-66 | 17 |
| | 54) | | |
| HFR3 | RVTITADKSTSTAYMELSSLRSEDTAV | 67-98 | 32 |
| | YYCAR (SEQ ID NO: 55) | | |
| CDR-H3 | GAIYDGYDVLDN (SEQ ID NO: 56) | 99-110 | 12 |
| HFR4 | WGQGTLVTVSS (SEQ ID NO: 57) | 111-121 | 11 |

(10) The linker sequence between heavy chain and light chain of BCMA-1 single-chain variable region is:

(SEQ ID NO: 10)
GSTSGSGKPGSGEGSTKG

(11) The linker sequence between heavy chain and light chain of BCMA-20, BCMA-CA8, and BCMA-MO6 single-chain variable region is:

(SEQ ID NO: 11)
GGGGSGGGGSGGGGS

(12) Sequence of hinge region and linker region:

(SEQ ID NO: 12)
FVPVFLPAKPTTTPAPRPPTPAPTIASQPLSLRPEACRPAAGGAVHTRG
LDFACD

(13) The transmembrane region is a transmembrane region sequence of CD8 (CD8TM) antigen:

(SEQ ID NO: 13)
IYIWAPLAGTCGVLLLSLVITLYC

(14) The co-stimulatory factor signal region is derived from the sequence of 4-1BB cytoplasmic signaling motif:

(SEQ ID NO: 14)
KRGRKKLLYIFKQPFMRPVQTTQEEDGCSCRFPEEEEGGCEL

(15) The signaling region of CD3 zeta is derived from the sequence of immunoreceptor tyrosine-based activation motif (ITAM) of CD3zeta in the TCR complex:

(SEQ ID NO: 15)
RVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTAT
KDTYDALHMQALPPR

In certain embodiments, the nucleic acid encoding the CAR (derived from the BCMA-20 antibody) may have the following sequence (SEQ ID NO: 58):

atggccttaccagtgaccgccttgctcctgccgctggccttgctgctcc
acgccgccaggccggacatccagatgacccagtccccctcctccctgtc
cgcctccgtgggcgaccgggtgaccatcacctgccgggcctcccagggc
atctccaactacctgaactggtaccagcagaagcccggcaaggccccca
agcccctgatctactacacctccaacctgcagtccggcgtgccctcccg
gttctccggctccggctccggcaccgactacaccctgaccatctcctcc
ctgcagcccgaggacttcgccacctactactgcatgggccagaccatct
cctcctacaccttcggccagggcaccaagctggagatcaagggtggcgg
tggctcgggcggtggtgggtcgggtggcggcggatctgaggtgcagctg
gtggagtccggcggcggcctggtgcagcccggcggctccctgcggctgt
cctgcgccgcctccggcttcaccttctccaacttcgacatggcctgggt
gcggcaggcccccggcaagggcctggtgtgggtgtcctccatcaccacc
ggcgccgaccacgccatctacgccgactccgtgaagggccggttcacca
tctcccgggacaacgccaagaacaccctgtacctgcagatgaactccct
gcgggccgaggacaccgccgtgtactactgcgtgcggcacggctactac
gacggctaccacctgttcgactactggggccagggcaccctggtgaccg
tgtcctccttcgtgccggtcttcctgccagcgaagcccaccacgacgcc
agcgccgcgaccaccaacaccggcgcccaccatcgcgtcgcagcccctg
tccctgcgcccagaggcgtgccggccagcggggggggcgcagtgcacac
gagggggctggacttcgcctgtgatatctacatctgggcgcccttggcc
gggacttgtggggtccttctcctgtcactggttatcaccctttactgca
aacggggcagaaagaaactcctgtatatattcaaacaaccatttatgag
accagtacaaactactcaagaggaagatggctgtagctgccgatttcca
gaagaagaagaaggaggatgtgaactgagagtgaagttcagcaggagcg
cagacgcccccgcgtaccagcagggccagaaccagctctataacgagct
caatctaggacgaagagaggagtacgatgttttggacaagagacgtggc
cgggaccctgagatggggggaaagccgagaaggaagaaccctcaggaag
gcctgtacaatgaactgcagaaagataagatggcggaggcctacagtga
gattgggatgaaaggcgagcgccggaggggcaaggggcacgatggcctt
taccagggtctcagtacagccaccaaggacacctacgacgcccttcaca
tgcaggccctgccccctcgctaa

In certain embodiments, the CAR (derived from the BCMA-20 antibody) may have the following amino acid sequence (SEQ ID NO: 59):

MALPVTALLLPLALLLHAARPDIQMTQSPSSLSASVGDRVTITCRASQG
ISNYLNWYQQKPGKAPKPLIYYTSNLQSGVPSRFSGSGSGTDYTLTISS
LQPEDFATYYCMGQTISSYTFGQGTKLEIKGGGGSGGGGSGGGGSEVQL
VESGGGLVQPGGSLRLSCAASGFTFSNFDMAWVRQAPGKGLVWVSSITT
GADHAIYADSVKGRFTISRDNAKNTLYLOMNSLRAEDTAVYYCVRHGYY
DGYHLFDYWGQGTLVTVSSFVPVFLPAKPTTTPAPRPPTPAPTIASQPL
SLRPEACRPAAGGAVHTRGLDFACDIYIWAPLAGTCGVLLLSLVITLYC
KRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALH
MQALPPR

In certain embodiments, the nucleic acid encoding the CAR (derived from the BCMA-CA8 antibody) may have the following sequence (SEQ ID NO: 60):

atggccttaccagtgaccgccttgctcctgccgctggccttgctgctcc
acgccgccaggccggatatccagctgacccagaccacaagcagcctgag
cgcctccctgggcgacagggtgaccattagctgcagcgccagccaggac
atcagcaactacctgaactggtaccagcagaagcccgacggcaccgtgg
agctcgtgatctactacacctccaacctgcacagcggcgtgcccagcag
gttctctggcagcggcagcggcaccgactacagcctgaccatcggctat
ctggagcccgaggacgtcgccacctactactgccagcagtacaggaagc
tgccctggaccttcggcggaggctctaagctggagattaagcgtggtgg
cggtggctcgggcggtggtgggtcgggtggcggcggatctgaggtgcag
ctgcagcagagcggcgccgtgctggccaggcccggagctagcgtgaaga
tgagctgcaagggcagcggctacaccttcaccaactactggatgcactg
ggtgaaacagaggcccggccagggactggagtggatcggcgccacctac
aggggccacagcgacacctactacaaccagaagttcaagggcaaggcca
agctgaccgccgtgacctcaaccagcaccgcctacatggaactgagcag
cctgaccaacgaggacagcgccgtctattactgcaccaggggcgccatc
tacaacggctacgacgtgctggacaattggggccagggaacactagtga
ccgtgtccagcttcgtgccggtcttcctgccagcgaagcccaccacgac
gccagcgccgcgaccaccaacaccggcgcccaccatcgcgtcgcagccc
ctgtccctgcgcccagaggcgtgccggccagcggcggggggcgcagtgc
acacgagggggctggacttcgcctgtgatatctacatctgggcgccctt
ggccgggacttgtggggtccttctcctgtcactggttatcaccctttac
tgcaaacggggcagaaagaaactcctgtatatattcaaacaaccattta
tgagaccagtacaaactactcaagaggaagatggctgtagctgccgatt
tccagaagaagaagaaggaggatgtgaactgagagtgaagttcagcagg
agcgcagacgcccccgcgtaccagcagggccagaaccagctctataacg
agctcaatctaggacgaagagaggagtacgatgttttggacaagagacg
tggccgggaccctgagatggggggaaagccgagaaggaagaaccctcag
gaaggcctgtacaatgaactgcagaaagataagatggcggaggcctaca
gtgagattgggatgaaaggcgagcgccggaggggcaaggggcacgatgg
cctttaccagggtctcagtacagccaccaaggacacctacgacgccctt
cacatgcaggccctgccccctcgctaa

In certain embodiments, the CAR (derived from the BCMA-CA8 antibody) may have the following amino acid sequence (SEQ ID NO: 61):

MALPVTALLLPLALLLHAARPDIQLTQTTSSLSASLGDRVTISCSASQD
ISNYLNWYQQKPDGTVELVIYYTSNLHSGVPSRFSGSGSGTDYSLTIGY
LEPEDVATYYCQQYRKLPWTFGGGSKLEIKRGGGGSGGGGSGGGGSEVQ
LQQSGAVLARPGASVKMSCKGSGYTFTNYWMHWVKQRPGQGLEWIGATY
RGHSDTYYNQKFKGKAKLTAVTSTSTAYMELSSLTNEDSAVYYCTRGAI
YNGYDVLDNWGQGTLVTVSSFVPVFLPAKPTTTPAPRPPTPAPTIASQP
LSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDAL
HMQALPPR

In certain embodiments, the nucleic acid encoding the CAR (derived from the BCMA-MO6 antibody) may have the following sequence (SEQ ID NO: 62):

atggccttaccagtgaccgccttgctcctgccgctggccttgctgctcc
acgccgccaggccggacatccagatgacccagagccctagctcactgag
cgccagcgtgggcgacagggtgaccattacctgctccgccagccaggac
atcagcaactacctgaactggtaccagcagaagcccggcaaggccccca
agctgctgatctactacacctccaacctgcactccggcgtgcccagcag
gttcagcggaagcggcagcggcaccgatttcaccctgaccatctccagc
ctgcagcccgaggacttcgccacctactactgccagcagtacaggaagc
tcccctggactttcggccagggcaccaaactggagatcaagcgtggtgg
cggtggctcgggcggtggtgggtcgggtggcggcggatctcaggtgcag
ctggtccagagcggcgccgaagtgaagaagcccggcagctccgtgaaag
tgagctgcaaggccagcggcggcaccttcagcaactactggatgcactg
ggtgaggcaggcccccggacagggcctggagtggatgggcgccacctac
aggggccacagcgacacctactacaaccagaagttcaagggccgggtga
ccatcaccgccgacaagagcaccagcaccgcctacatggaactgagcag
cctcaggagcgaggacaccgctgtgtattactgcgccaggggcgccatc
tacgacggctacgacgtgctggacaactggggccagggcacactagtga
ccgtgtccagcttcgtgccggtcttcctgccagcgaagcccaccacgac
gccagcgccgcgaccaccaacaccggcgcccaccatcgcgtcgcagccc
ctgtccctgcgcccagaggcgtgccggccagcggggggggcgcagtgca
cacgagggggctggacttcgcctgtgatatctacatctgggcgcccttg
gccgggacttgtggggtccttctcctgtcactggttatcaccctttact
gcaaacggggcagaaagaaactcctgtatatattcaaacaaccatttat
gagaccagtacaaactactcaagaggaagatggctgtagctgccgattt
ccagaagaagaagaaggaggatgtgaactgagagtgaagttcagcagga
gcgcagacgcccccgcgtaccagcagggccagaaccagctctataacga
gctcaatctaggacgaagagaggagtacgatgttttggacaagagacgt
ggccgggaccctgagatggggggaaagccgagaaggaagaaccctcagg
aaggcctgtacaatgaactgcagaaagataagatggcggaggcctacag
tgagattgggatgaaaggcgagcgccggaggggcaaggggcacgatggc
ctttaccagggtctcagtacagccaccaaggacacctacgacgcccttc
acatgcaggccctgccccctcgctaa

In certain embodiments, the CAR (derived from the BCMA-BCMA-MO6 antibody) may have the following amino acid sequence (SEQ ID NO: 63):

MALPVTALLLPLALLLHAARPDIQMTQSPSSLSASVGDRVTITCSASQD
ISNYLNWYQQKPGKAPKLLIYYTSNLHSGVPSRFSGSGSGTDFTLTISS
LQPEDFATYYCQQYRKLPWTFGQGTKLEIKRGGGGSGGGGSGGGGSQVQ
LVQSGAEVKKPGSSVKVSCKASGGTFSNYWMHWVRQAPGQGLEWMGATY
RGHSDTYYNQKFKGRVTITADKSTSTAYMELSSLRSEDTAVYYCARGAI
YDGYDVLDNWGQGTLVTVSSFVPVFLPAKPTTTPAPRPPTPAPTIASQP
LSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDAL
HMQALPPR

Chimeric Antigen Receptor T Cells (CAR-T Cells)

As used herein, the terms “CAR-T cell”, “CAR-T”, and “CART”, may be used interchangeably.

The present disclosure relates to the construction of a chimeric antigen receptor structure targeting BCMA, a preparation method of a chimeric antigen receptor engineered T cell targeting BCMA, and activity identification thereof.

Vector

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The present disclosure also provides vectors in which the expression cassette of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the advantage over vectors derived from onco-retroviruses such as murine leukemia viruses in that they can transduce non-proliferating cells, such as hepatocytes. They also have the advantage of low immunogenicity.

In brief summary, the expression cassette or nucleic acid sequence is typically and operably linked to a promoter, and incorporated into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the present disclosure may also be used for nucleic acid immune and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, either constitutive promoters or inducible promoters may be used. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionein promoter, a glucocorticoid promoter, a progesterone promoter, and a tetracycline promoter.

In order to assess the expression of a CAR polypeptide or portions thereof, the expression vector to be introduced into a ceil can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In one embodiment, the vector is a lentiviral vector.

Compositions

The disclosure provides a composition comprising the immune cell (e.g., CAR-T cell), and a pharmaceutically acceptable carrier, diluent and/or excipient. In one embodiment, the composition is a liquid composition. For example, the composition is an injectable composition. In certain embodiments, the concentration of the CAR-T cells in the composition is 1×10³-1×10⁸ cells/ml, or 1×10⁴-1×10⁷ cells/ml.

In one embodiment, the composition may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The composition may be formulated for intravenous administration.

Therapeutic Application

The disclosure comprises therapeutic applications using cells (e.g., T cells) transduced with a lentiviral vector (LV) encoding the expression cassette of the disclosure. The transduced T cells can target the tumor cell marker BCMA, synergistically activate T cells, and cause T cell immune responses, thereby significantly increasing the killing efficiency against tumor cells.

Thus, the present disclosure also provides a method for stimulating a T cell-mediated immune response to a target cell population or tissue in a mammal comprising the step of administering to the mammal a CAR-T cell of the disclosure.

In one embodiment, the present disclosure comprises a class of cell therapies, wherein T cells from autologous patient (or heterologous donor) are isolated, activated and genetically modified to generate CAR-T cells, and then injected into the same patient. The probability of graft versus host disease in the way is extremely low, and antigens are recognized by T cells in a non-MHC-restricted manner. In addition, one CAR-T can treat all cancers that express the antigen. Unlike antibody therapies, CAR-T cells are able to replicate in vivo resulting in long-term persistence that can lead to sustained tumor control

In one embodiment, the CAR-T cells of the disclosure can undergo robust in vivo T cell expansion and can persist for an extended amount of time. In addition, the CAR mediated immune response may be part of an adoptive immunotherapy approach in which CAR-modified T cells induce an immune response specific to the antigen binding moiety in the CAR. For example, an anti-BCMA CAR-T cell elicits an immune response specific against cells expressing BCMA.

Although the data disclosed herein specifically disclose lentiviral vector comprising BCMA scFv, hinge and transmembrane domain, and 4-1BB and CD3ζ signaling domains, the disclosure should be construed to include any number of variations for each of the components of the construct as described elsewhere herein.

Cancers that may be treated include tumors that are unvascularized or largely unvascularized, and tumors that are vascularized. Cancers may include non-solid tumors (such as hematological tumors, for example, leukemias and lymphomas) or solid tumors. Types of cancers to be treated with the CARs include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, mesothelioma, malignant lymphoma, pancreatic cancer and ovarian cancer.

The CAR-modified T cells may also serve as a type of vaccine for ex vivo immunization and/or in vivo therapy in a mammal. Preferably, the mammal is a human.

With respect to ex vivo immunization, at least one of the following occurs in vitro prior to administering the cell into a mammal: i) expanding the cells, ii) introducing a nucleic acid encoding a CAR to the cells, and/or iii) cryopreservation of the cells.

Ex vivo procedures are well known in the art and are discussed more fully below. Briefly, cells are isolated from a mammal (such as a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

In addition to using a cell-based vaccine in terms of ex vivo immunization, the present disclosure also provides compositions and methods for in vivo immunization to elicit an immune response directed against an antigen in a patient.

The present disclosure provides methods for treating tumors comprising administering to a subject in need thereof, a therapeutically effective amount of the CAR-modified T cells.

The CAR-modified T cells of the present disclosure may be administered either alone, or as a pharmaceutical composition in combination with diluents and/or with other components such as IL-2, IL-17 or other cytokines or cell populations. Briefly, pharmaceutical compositions of the present disclosure may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, or 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous injection, or intraperitoneally. In one embodiment, the T cell compositions of the present disclosure are administered to a patient by intradermal or subcutaneous injection. In another embodiment, the T cell compositions of the present disclosure may be administered by intravenous injection. The compositions of T cells may be injected directly into a tumor, lymph node, or site of infection.

In certain embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the disclosure may be used in combination with chemotherapy, radiation, immunosuppressive agents, such as cyclosporin, azathioprine, methotrexate, mycophenolate, and FK506, antibodies, or other immunotherapeutic agents. In a further embodiment, the cell compositions of the present disclosure are administered to a patient in conjunction with (e.g., before, simultaneously or following) bone marrow transplantation, or the use of chemotherapy agents such as, fludarabine, external-beam radiation therapy (XRT), cyclophosphamide. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

The dosage of the above treatments to be administered to a patient will vary with the precise nature of the condition being treated and the recipient of the treatment. The scaling of dosages for patient administration can be performed according to art-accepted practices. In general, 1×10⁶ to 1×10¹⁰ of the modified T cells of the disclosure (e.g., CAR-T-BCMA cells) can be applied to patients by means of, for example, intravenous infusion each treatment or each course of treatment.

The main advantages of the present disclosure include:
(a) As for the chimeric antigen receptor of the present disclosure, the extracellular antigen binding domain is a specific anti-BCMA scFv; the CARs have shown a great ability of killing tumor cells with low cytotoxicity and low side effects.
(b) The chimeric antigen receptor provided by the disclosure can achieve stable expression and membrane localization of CAR protein after T cells are infected by viral vectors (e.g., lentiviruses) carrying the CAR gene.
(c) The CAR-modified T cells of the present disclosure have a longer survival time in vivo and strong anti-tumor effect. The scFv used in the present disclosure may be a humanized or human-derived antibody, and is less likely to produce specific immunological rejection.

The present disclosure will be further illustrated below with reference to the specific examples. It is to be understood that these examples are for illustrative purposes only and are not intended to limit the scope of the invention. For the experimental methods in the following examples the specific conditions of which are not specifically indicated, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified. Percentages and parts are by weight unless otherwise stated.

### Example 1 Construction of Lentiviral Expression Vector

The full-length DNA synthesis and cloning were commissioned by Shanghai Boyi Biotechnology Co., Ltd to achieve the construction of coding plasmids. The pWPT lentiviral vector was selected as a cloning vector, and the cloning sites were BamH I and Sal I sites. The specific sequence is as described above.

### Example 2 Preparation of CAR-T Cell

(1) Mononuclear cells (PBMCs) were isolated from venous blood of healthy people by density gradient centrifugation.
(2) On day 0, PBMCs were seeded in a cell culture flask previously coated with CD3 monoclonal antibody (OKT3) at a final concentration of 5 μg/mL and Retronectin (purchased from TAKARA) at a final concentration of 10 μg/mL. The medium was GT-551 cell culture medium containing 1% human albumin. Recombinant human interleukin 2 (IL-2) was added to the medium at a final concentration of 1000 U/mL. The cells were cultured in an CO₂ incubator with a saturated humidity of 5% at 37° C.
(3) On day 1, the supernatant of the cultured PBMCs was slowly aspirated and discarded. New GT-551 cell culture medium containing 1% human albumin was added, and recombinant human interleukin 2 (IL-2) was added to the medium at a final concentration of 1000 U/mL. The cells were continuously cultured in an CO₂ incubator with a saturated humidity of 5% at 37° C.
(4) On day 3, fresh medium, concentrated and purified CAR-BCMAs lentivirus solution, protamine sulfate (12 ug/ml), and IL-2 (at a final concentration of 1000 U/mL) were added. After 12 hours of infection in a 5% CO₂ incubator at 37° C., the culture medium was discarded, fresh medium was added, and cultivation was continued in a 5% CO₂ incubator at 37° C.
(5) Starting from day 6, CAR-BCMAs cells can be taken for the corresponding activity assay.

### Example 3 Detection of the Integration Rate of the CAR Gene in the T Cell Genome and the Expression Level of the Encoded Protein Thereof on the Membrane Surface

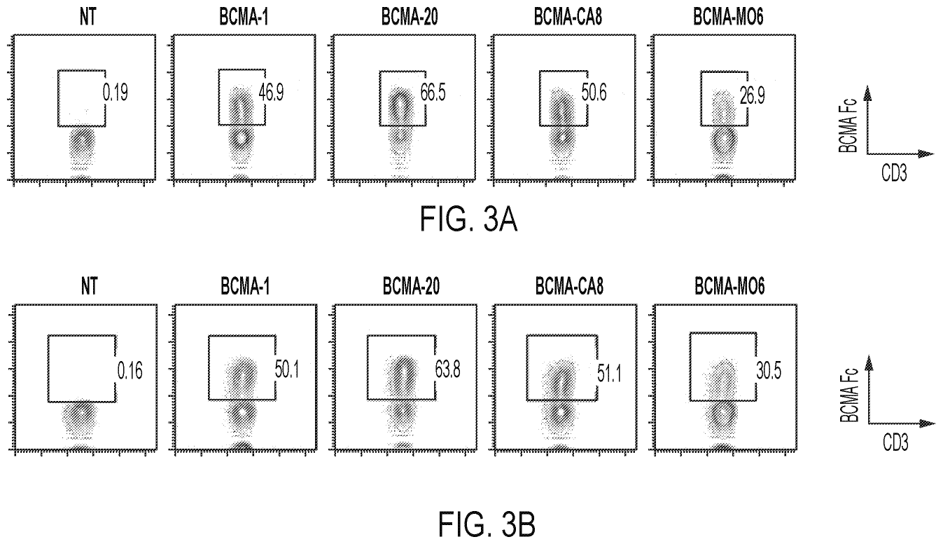

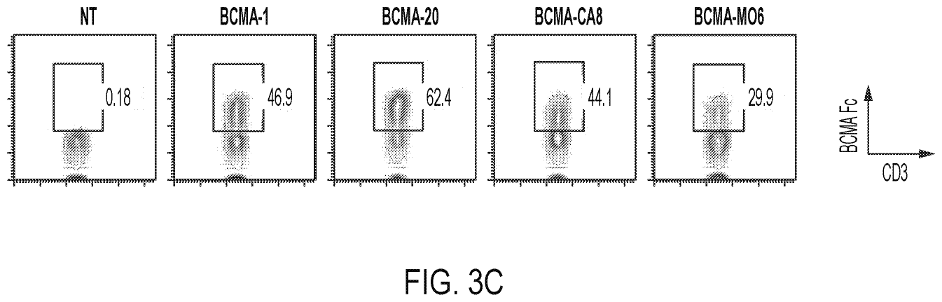

0.5×10⁶ of CART-BCMAs cell samples cultured on day 7 (FIG. 3A), day 21 (FIG. 3B) and day 29 (FIG. 3C) in Example 2 were taken, respectively. The expression level of CAR-BCMA protein on the surface of T cell membrane was analyzed by flow cytometry after Fc fragment staining of recombinant human BCMA protein.

The result is shown in FIGS. 3A, 3B and 3C, and four CAR structures designed in the present disclosure can be expressed in their corresponding modified T cells and complete the cell membrane surface localization.

### Example 4 Detection of the In Vitro Activation Ability of CAR-BCMAs

Cell activation level indicator proteins CD137 and IFNγ was detected using CART-BCMAs cells cultured on day 7 in Example 2. 1×10⁵ of CART-BCMA cells cultured on day 7 were cultured respectively with BCMA-positive K562-BCMA+E7 tumor cell line and BCMA-negative K562 tumor cell line, or without tumor cells, in 200 μl GT-551 medium for 18 h at a ratio of 1:1. Then the expression level of CD137 on the surface of T cell membrane was detected by flow cytometry and the secretion level of IFNγ in the culture supernatant was detected by ELISA.

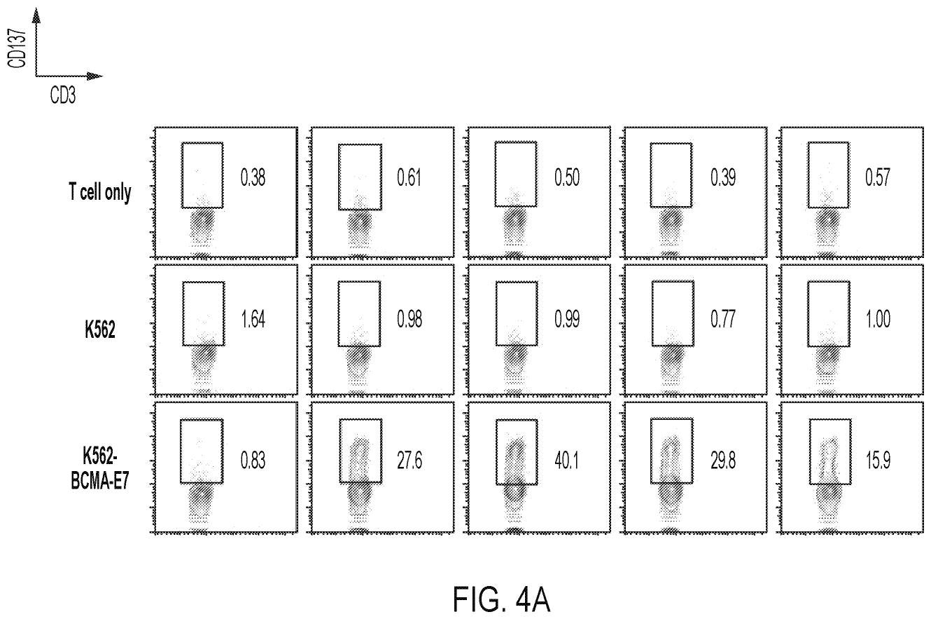

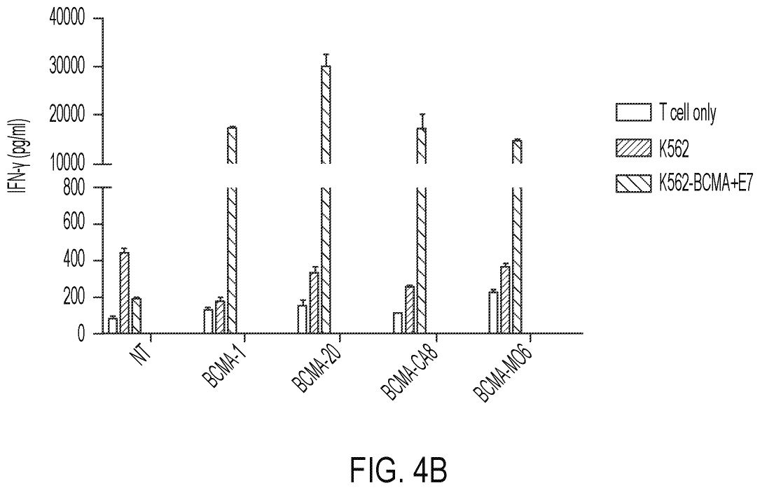

The results are shown in FIG. 4A and FIG. 4B, the expression of CD137 was detected on the surface of four CART cells, and the expression of IFNγ was detected in the culture supernatant. Among them, CAR-BCMA-20 shows best CD137 activation level and IFNγ release level. CART-BCMA-MO6 which is constructed based on humanized MO6 antibody sequence shows a weaker level of CD137 activation but a higher level of IFNγ release than CART-BCMA-CA8 which is constructed based on mouse antibody sequence.

Additionally, C-CAR088 induced higher levels of IFN-γ release (FIG. 4B) and CD137 expression (FIG. 4A) in BCMA-positive tumor cells, compared to CARs based on BCMA-1, BCMA-CA8 and BCMA-MO6.

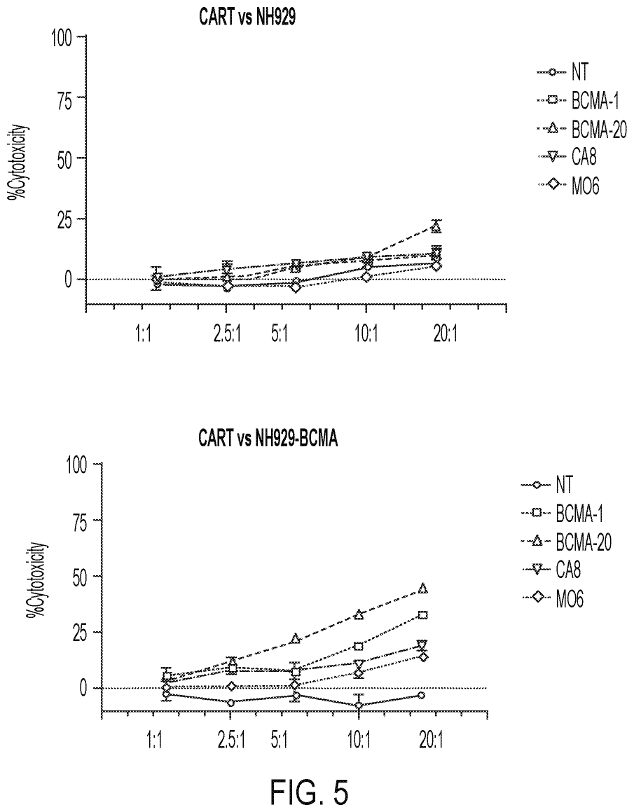

CART-BCMA-20 induced greater apoptosis of BCMA-positive tumor cells than CART-BCMA-1 (positive control), CART-BCMA-MO6 and CART-BCMA-CA8 (FIG. 5).

### Example 5 Detection of Advanced Apoptosis Activity of Tumor Cells Induced by CART-BCMAs Cells

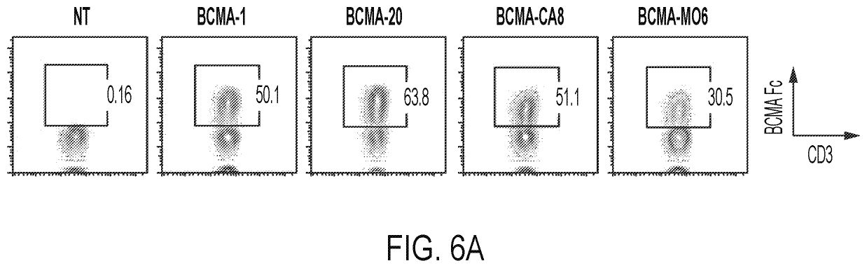

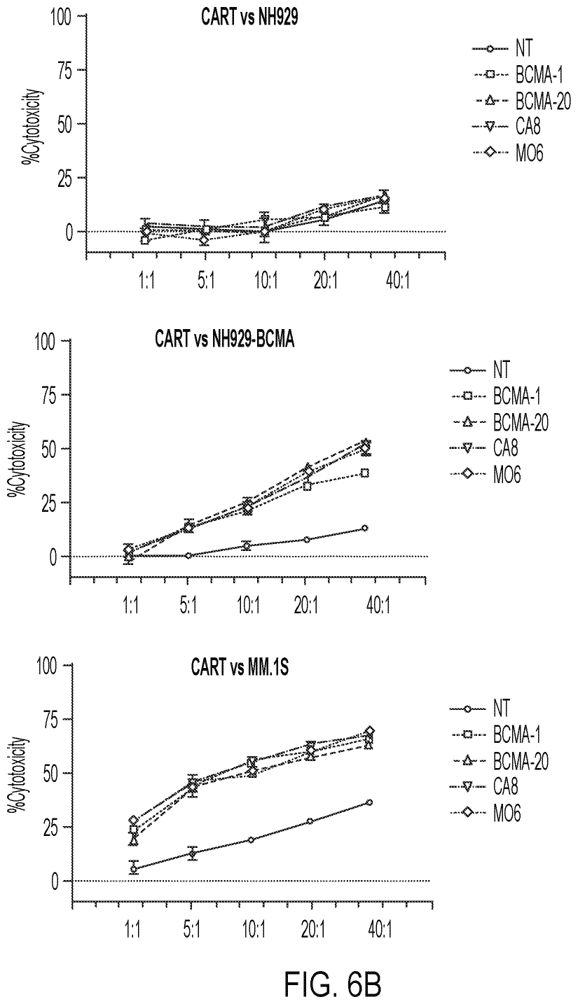

(1) The CART-BCMAs cells cultured on day 17 in Example 2 were mixed respectively with 1×10⁴ CFSE-labeled BCMA-negative cells (NH929) or BCMA-positive self-constructed cells (NH929-BCMA overexpressing tumor cell line) at a ratio of 1:1, 2.5:1, 5:1, 10:1, 20:1 (as shown in FIG. 5). The mixed cells were co-cultured in 100 μl GT-551 medium for 4 h, and then stained with 100 μl 25% PI dye for 15 min. The proportion of PI positive cells in CFSE positive cells was analyzed by flow cytometry.
(2) The CART-BCMAs cells cultured on day 22 in Example 2 were mixed respectively with 1×10⁴ CFSE-labeled BCMA-negative cells (NH929), BCMA-positive self-constructed cells (NH929-BCMA overexpressing tumor cell line) or MM.1S cell line naturally expressed BCMA at a ratio of 1:1, 5:1, 10:1, 10:1, 40:1 (as shown in FIG. 6B). The mixed cells were co-cultured in 100 μl GT-551 medium for 4 h, and then stained with 100 μl 25% PI dye for 15 min. The proportion of PI positive cells in CFSE positive cells was analyzed by flow cytometry.

The results are shown in FIG. 5 and FIGS. 6A and 6B, all four CART cells can induce apoptosis of BCMA-positive tumor cells. Among them, CART-BCMA-20 can induce advanced apoptosis of BCMA-positive tumor cells better than CART-BCMA-1. The ability of CART-BCMA-MO6 and CART-BCMA-CA8 to induce advanced apoptosis of BCMA-positive tumor cells is similar.

### Example 6 Inhibition of CART-BCMAs on RPMI-8226 Myeloma Xenograft Model

RPMI-8226 cells in logarithmic growth phase were collected, and 4.0×10⁶ tumor cells were inoculated subcutaneously in the right back of 6-8 week old B-NDG mice. When the tumor volume reached about 120 mm³, the animals were randomly divided into 4 groups according to tumor volume, so that the tumor volume difference of each group was less than 10% of the mean value. Then the solvent control, 7.5×10⁶ NT and 7.5×10⁶ CART-BCMAs cells were injected through tail vein respectively.

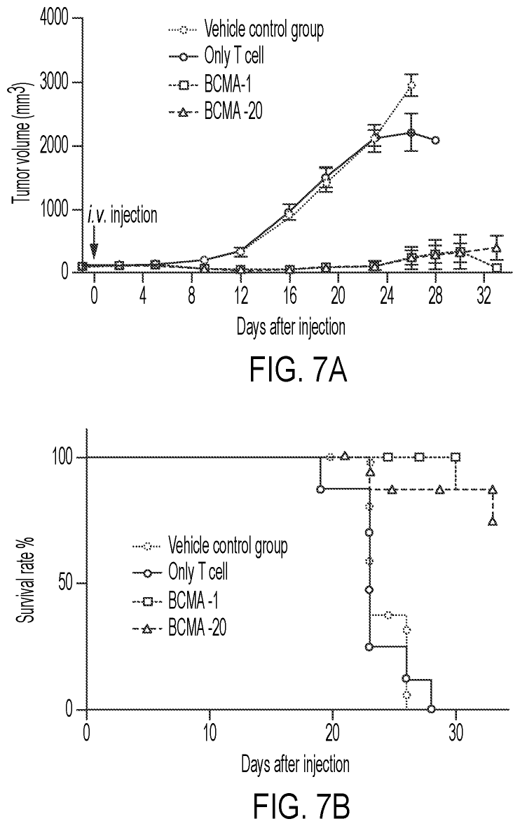

The results are shown in FIGS. 7A and 7B, compared with the control group, single injection of CART-BCMA-1 and CART-BCMA-20 through tail vein can effectively inhibit the growth of human myeloma RPMI-8226 cells (relative tumor proliferation rate % T/CRTV≤40%, P<0.05), and can significantly prolong the survival time of human myeloma-bearing mice (median survival of the control group is 23 days, median survival of the CART-BCMAs treatment group >33 days). There was no significant difference in tumor proliferation rate and median survival between the mice treated with CART-BCMA-1 and CART-BCMA-20.

For in vivo studies, B-NDG mice were xenografted with human myeloma RPMI-8226 cells. When the tumor volume reached about 120 mm³, the solvent control, non-transfected (NT) T cells (a negative control) or CART-BCMAs cells were injected through the tail vein of the mice.

Compared with the control group, a single injection of CART-BCMA-1 and CART-BCMA-20 effectively inhibited the growth of the myeloma cells, and significantly prolonged the survival time of human myeloma-bearing mice: median survival of the CART-BCMA-treated groups was >33 days, while median survival of the control group was 23 days. There was no significant difference in the tumor proliferation rate and median survival between the mice treated with CART-BCMA-1 and the mice treated with CART-BCMA-20 (FIGS. 7A and 7B).

The results suggest that BCMA-20 (C-CAR088) had high in vivo anti-tumor efficacy.

### Comparative Example

In the screening process of the chimeric antigen receptor of the present application, the inventors tested a large number of candidate sequences, which are illustrated below with examples.

The antibodies to be screened include: BCMA-1, BCMA-2, BCMA-69, BCMA-72, BCMA-2A1, BCMA-1E1, BCMA-J22.9, BCMA-20, BCMA-CA8, and BCMA-MO6. Structures of chimeric antigen receptor targeting BCMA were constructed on the basis of the above antibodies. Among them, BCMA-1 and BCMA-2 are published Car-T sequences and used as a positive control for screening. CAR-T cells were prepared in the same way as in Example 2, and detected in the same way as in Examples 3 and 4.

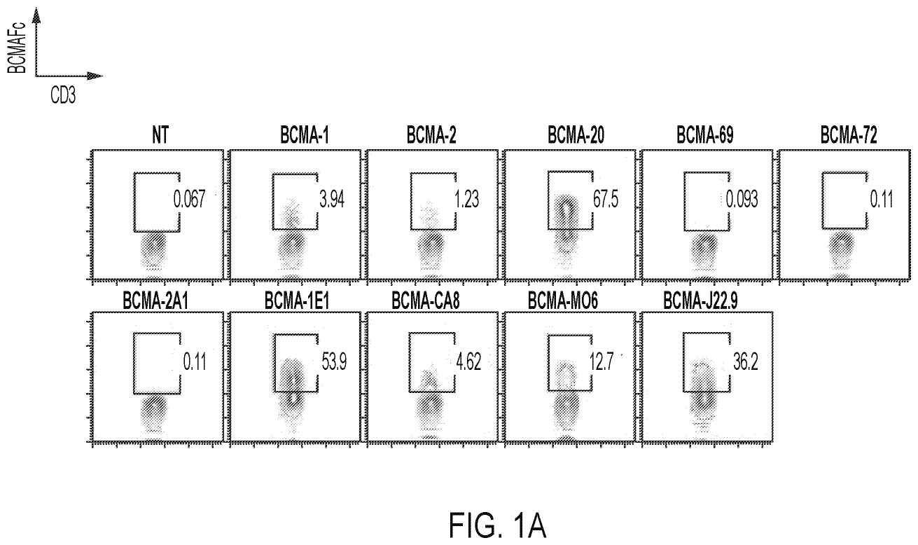

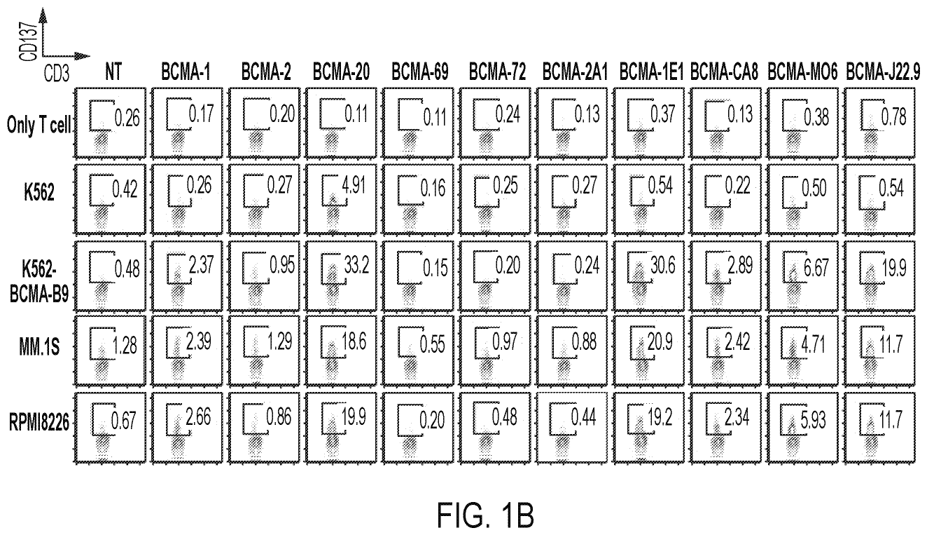

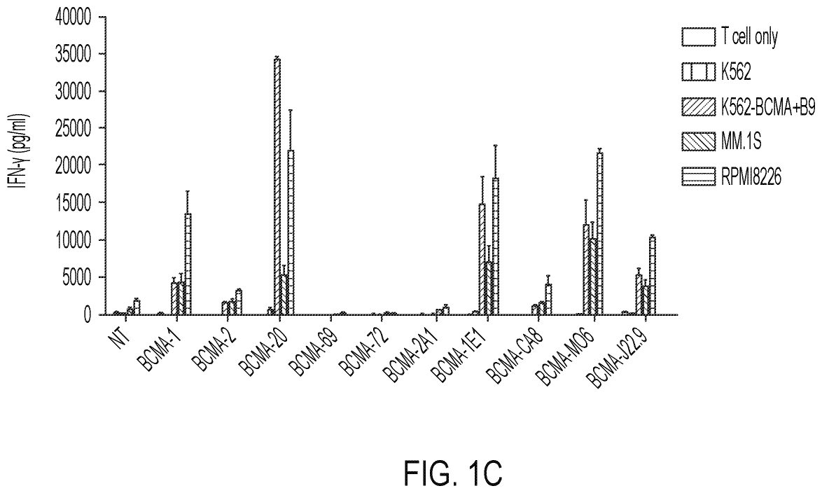

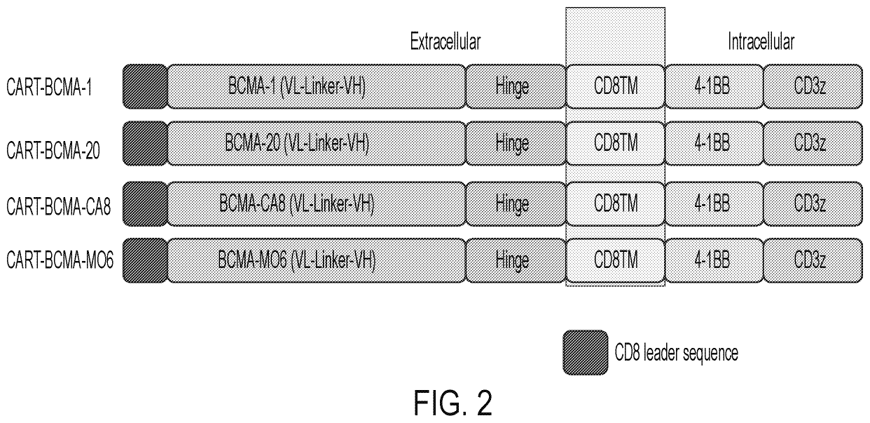

The results are shown in FIGS. 1A, 1B and 1C, which are the experimental results of two batches of CAR-T cells. The expression of Car-T was detected with BCMA-Fc fusion protein. It can be seen that there is a high expression in primary T cells, as seen in FIG. 1A. In FIG. 1B, it can be seen that BCMA-1, BCMA-20, BCMA-1E1, BCMA-CA8, BCMA-MO6, and BCMA-J22.9 can be activated by the BCMA antigen. FIG. 1C shows that the activated BCMA-1, BCMA-20, BCMA-1E1, BCMA-CA8, BCMA-MO6, and BCMA-J22.9 CAR-Ts can produce higher levels of IFN-γ. The results show that the CAR-T functions obtained by BCMA-1, BCMA-20, CA8 and MO6 are similar, so BCMA-20, CA8 and MO6CAR-T were further analyzed and studied.

### Example 7 Membrane Protein Array Experiment

The light chain variable region of SEQ ID NO: 1 and the heavy chain variable region of SEQ ID NO: 2 were used to prepare a single chain antibody B20-scFv-rabFc, and membrane protein array experiment was performed.

20 ug/mL of B20-scFv-rabFc was added to HEK293T cell array transiently transfected with 5344 membrane proteins, respectively. Flow cytometry found that, under this test condition, B20-scFv-rabFc may cross-recognize with TNFRSF17 (Q02223), MAG (P20916), CR2 (P20023), CXADR (P78310) and DDR2 (Q16832), wherein TNFRSF17, i.e., BCMA is the specific target of B20-scFv, and MAG, CR2, CXADR and DDR2 are suspected non-specific targets.

To further confirm whether the suspected target can cause the activation of CAR-T, CBM.BCMA CAR-T was co-cultured with 293T cells transfected with BCMA, CR2, CXADR, DDR2, and MAG, respectively. The IFNγ, TNF, IL-2 and other cytokines in co-culture supernatant were detected. 293T cells transfected with empty vector were used as a negative control, and 293T cells transfected with BCMA were used as a positive control.

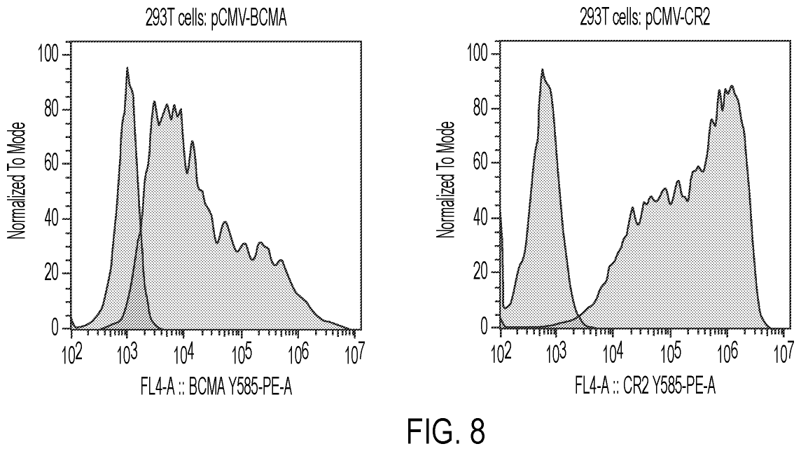

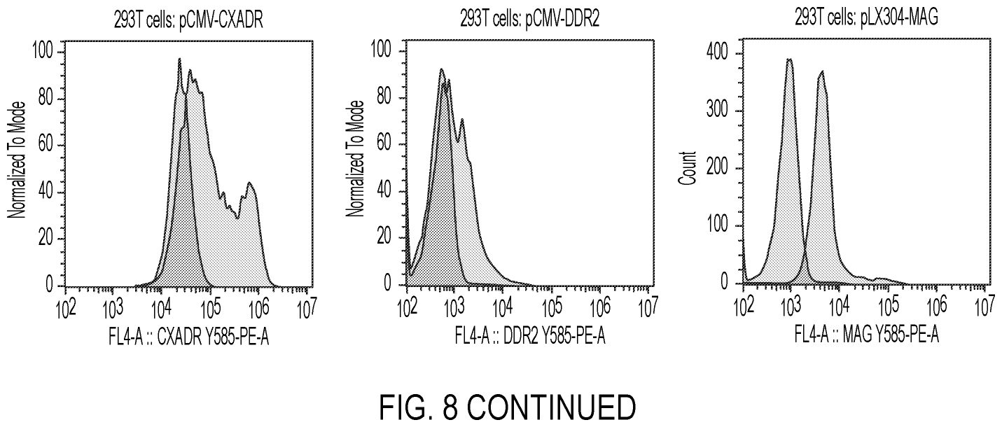

FIG. 8 shows results the expression of BCMA, CR2, CXADR, DDR2, and MAG in 293T cells after transfection of relevant plasmids by flow cytometry.

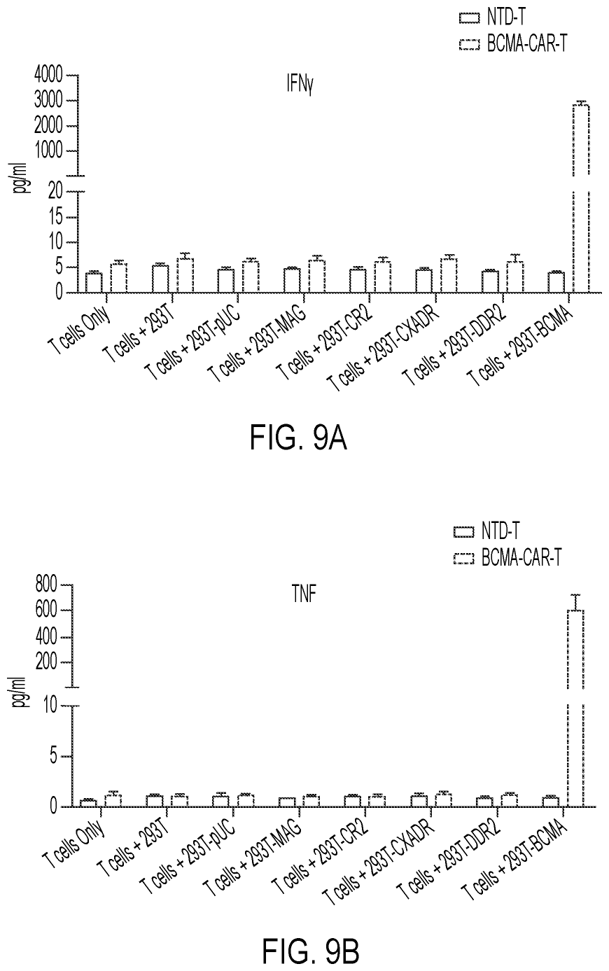

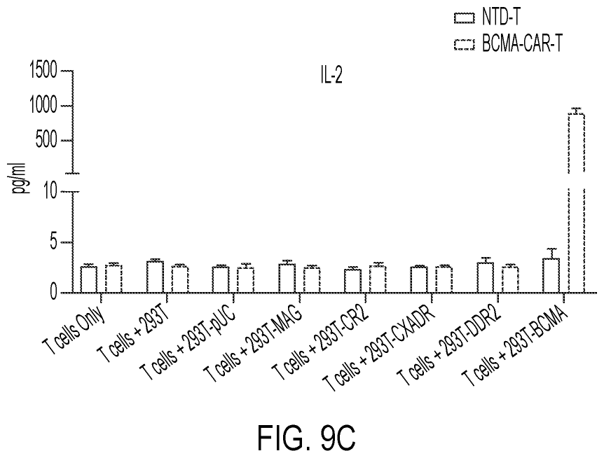

The cytokine detection results are shown in FIGS. 9A, 9B and 9C. Only the BCMA expressed on 293T cells can induce CBM. BCMA CAR-T cells to produce large amounts of IFNγ/TNF/IL-2, while the other four non-specific surface markers cannot activate CBM. BCMA CAR-T cells.

FIG. 8 and FIGS. 9A-9C show whole genome membrane proteome array and validation. TNFRSF17, MAG, CR2, CXADR and DDR2 were identified with moderate binding with B-20 scFv rabFc at the concentrations of 20.0 μg/mL. Only cells expressing TNFRSF17(BCMA) can induce BCMA-CAR-T cells producing a large number of cytokines (IFNγ/TNF/IL-2).

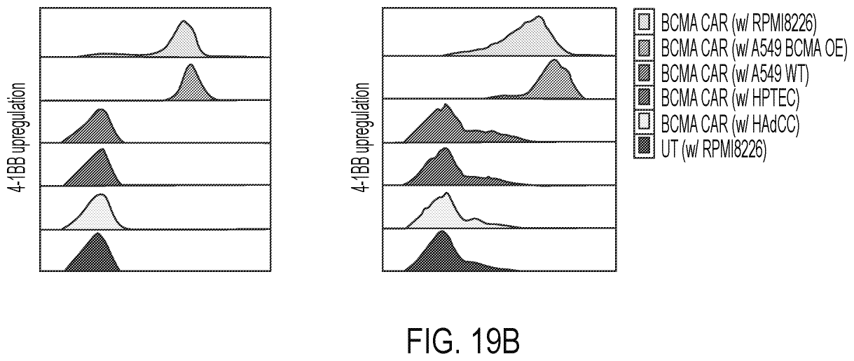

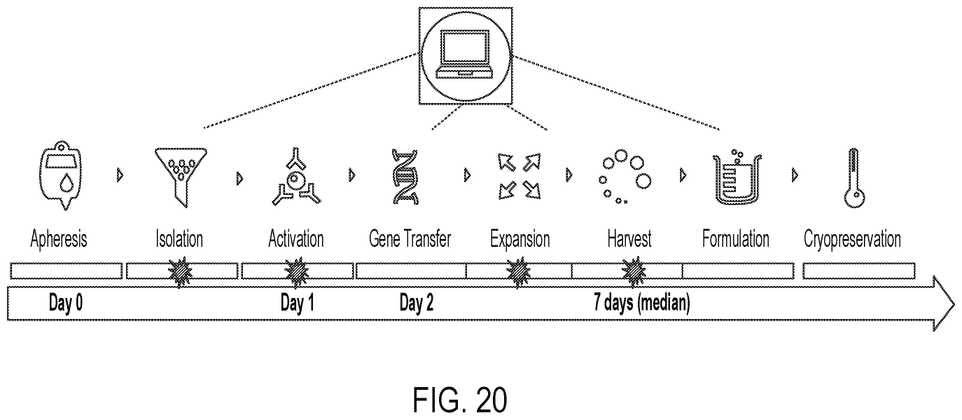

Specific membrane staining was observed on human lymphocytes in thymus, spleen, lymph nodes, bone marrow and scattered lymphocytes in thyroid gland, adrenal gland at the concentrations of 20.0 μg/mL (Table 1 and FIGS. 19A-19B).

TABLE 1

| Staining site | Tissue | 5.0 μg/mL | 20.0 μg/mL |
|---|---|---|---|
| Nucleus | Esophagus, mucosal | 1/3^{a}, +^{b} | 1/3, + |
| | layer | | |
| Cytoplasm | Stomach, mucosal | 1/3, + | 2/3, + |
| | layer | | |
| | Kidney, cortex | 1/3, + | 2/3, + |
| | Bladder, mucosal | − | 1/3, + |
| | layer | | |
| Membrane | Thyroid | − | 3/3, + |
| | Thymus, cortex, | 3/3, + | 3/3, + |
| | medulla | | |
| | Spleen, splenic cord | 2/3, + | 3/3, +~++ |
| | Lymph node, medulla | 3/3, + | 3/3, + |
| | Adrenal gland, cortex | − | 3/3, + |
| | Bone marrow | − | 3/3, + |

^{a}number of positive staining in 3 donors.
^{b}staining intensity of positive cells and percentage of positive cells in the cells of the same type.
“−” No stained cells,
“+” weakly positive staining, <10% cells are stained,
“++” moderately positive staining, 11%-50% cells are stained.

### Example 8 In Vitro Anti-Tumor Activity of C-CAR088 Cells

The chimeric antigen receptor BCMA-20 (hereafter named C-CAR088) was selected for subsequent experiments. C-CAR088 cells, NT cells (non-transfected T cells, used as negative control) and positive control cells (Bluebird bb2121) were co-cultured with BCMA negative cells (NH929) or BCMA positive cells (NH929-BCMA) at different effect target ratios, The killing ability of each cell to the target cell was analyzed.

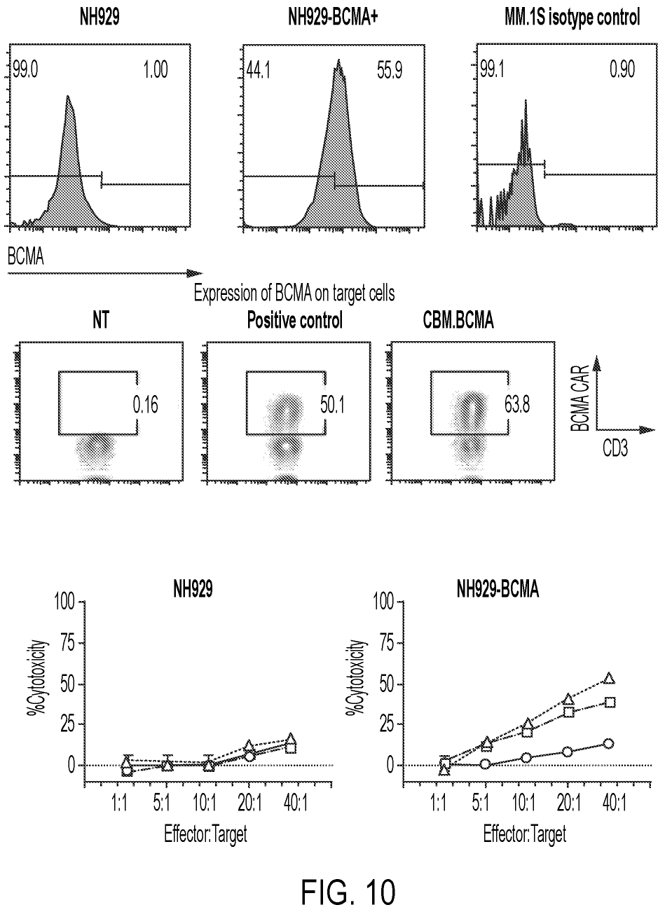

The results are shown in FIG. 10. C-CAR088 cells and positive control cells only have a strong killing effect on NH929-BCMA cells, but no significant killing effect on negative target cells NH929. The non-transfected group (NT) also have no significant killing effect on the target cells.

T cells with C-CAR088, non-transfected (NT) T cells (a negative control) and positive control cells (Bluebird bb2121) were co-cultured with BCMA negative cells (NH929) or BCMA positive cells (NH929-BCMA) at different effect/target ratios. Then the cytotoxicity was assayed.

FIG. 10 shows that C-CAR088 T cells and the positive control demonstrated strong cytotoxicity on NH929-BCMA cells, but not NH929 cells.

### Example 9 Effect of Soluble BCMA on Cell Killing Activity

In the co-cultivation system of C-CAR088 cells with BCMA negative target cells A549 and BCMA positive target cells A549-BCMA-2E9, 100 ng/ml and 500 ng/ml soluble BCMA protein was added respectively to detect its effect on CD137 expression.

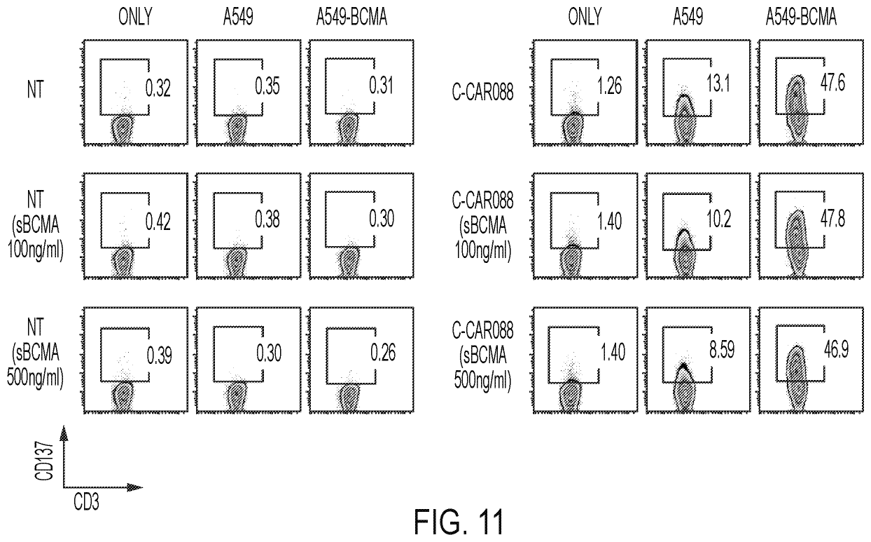

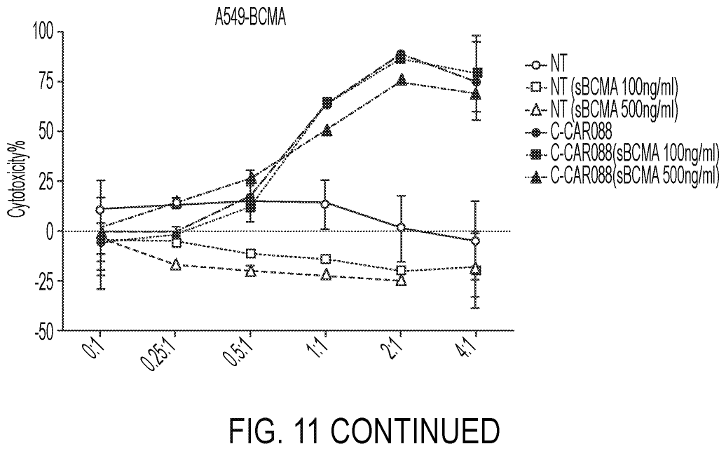

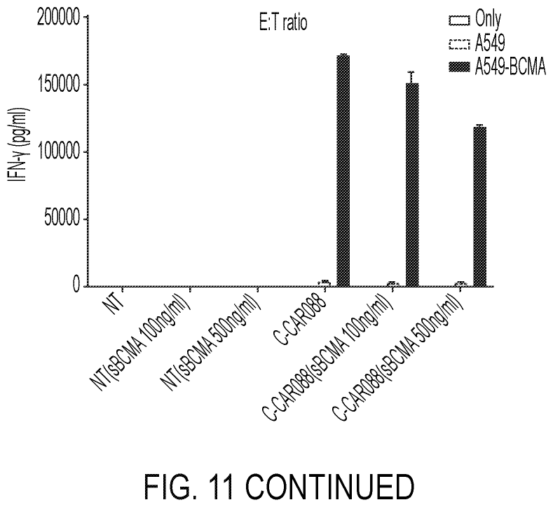

The results are shown in FIG. 11. Soluble BCMA protein have no effect on the upregulation of CD137 expression, indicating that soluble BCMA does not block the specific recognition of CAR and target antigen. In the test of in vitro cell killing ability and ELISA of IFN-γ release, the killing effect of C-CAR088 cells on target cells and the release of IFN-γ were reduced by 500 ng/ml soluble BCMA protein, but there was no significant difference. The above results indicate that the cell killing activity of C-CAR088 cells is basically unaffected by soluble BCMA

### Example 10 Dose Dependent Effect of C-CAR088 Cells

6 week old B-NDG mice (half male and half female) were selected and intraperitoneally injected with 2.5×10⁶ human multiple myeloma cells RPMI-8226. Mice with similar tumor burden were selected and divided into 5 groups, and were injected with 2.5×10⁶ C-CAR088 cells (low-dose group), 5×10⁶ C-CAR088 cells (medium-dose group), 1×10⁷ C-CAR088 cells (high-dose group), T cells and vehicle (with cryoprotectant (CBMG C-CFMC) as vehicle), respectively. The experiment lasted 54 days. During the experiment, the tumor burden of the mice was evaluated every 5 days. At the end of the experiment, the survival rate of the mice was calculated.

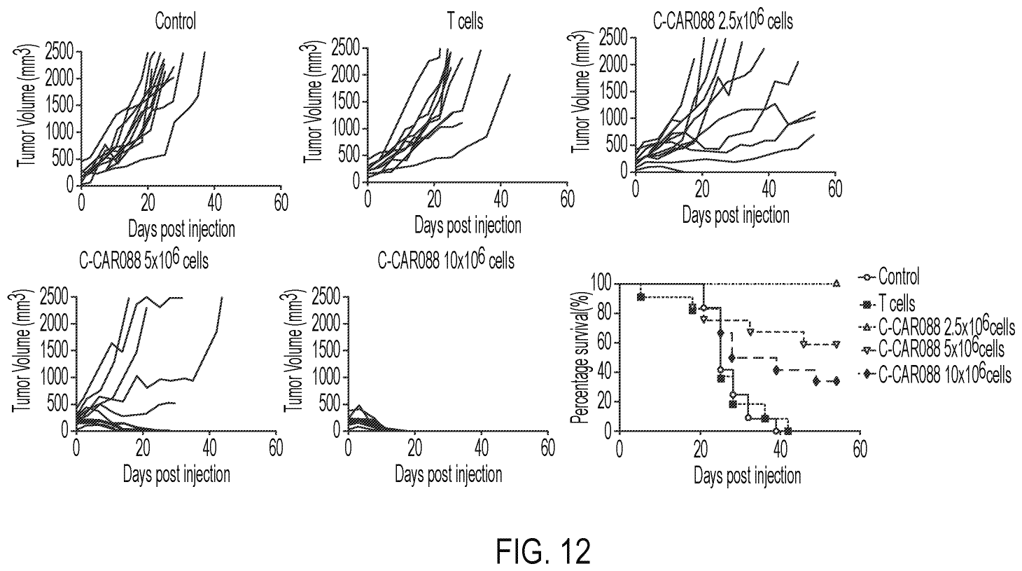

The results are shown in FIG. 12. Single administration of doses of 5×10⁶ cells/mouse and 1×10⁷ cells/mouse C-CAR088 cells can effectively inhibit the growth of B-NDG mouse xenograft tumor of human myeloma RPMI-8226 cell. The relative tumor proliferation rates were 6.12% and respectively (p<0.05). At the end of the experiment, the survival rate of tumor-bearing mice in the middle-dose group was 91.7% ( 11/12). No death occurred in the tumor-bearing mice in the high-dose group, and the survival rate was 100.0% (12/12), having a significant difference (p<0.05) compared with the vehicle group (survival rate 58.3%). The above results indicate that C-CAR088 exhibits dose dependent in vivo anti-tumor activity.

### Example 11 Phase I Clinical Study of C-CAR088

With the approval of the Ethics Committee, a total of 15 volunteers conducted phase I clinical trials. The key eligibility criteria for volunteer are as follows: patients with multiple myeloma aged 18-75 years old, MM cells express BCMA, have measurable MM, and have received 2 prior lines of therapy for MM and have received treatment with PI and IMiD, have adequate hepatic, renal, cardiac and hematopoietic function.

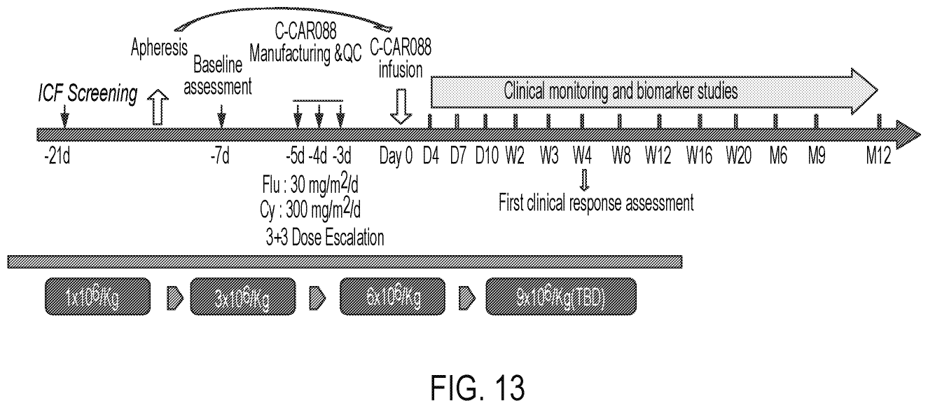

The experimental process is shown in FIG. 13.

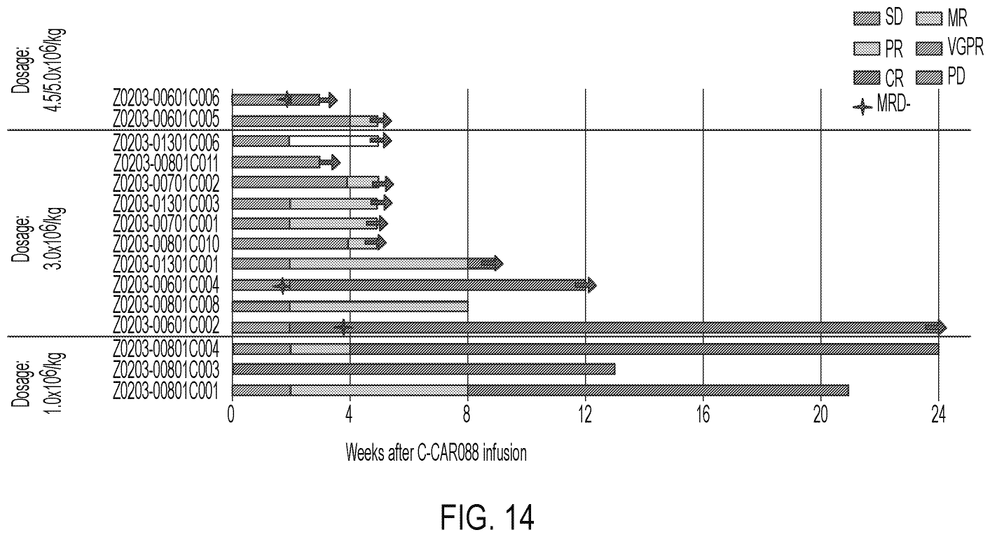

The treatment results are shown in FIG. 14 and Table 2. Two patients were evaluated at week 2, one SD and one CR. The remaining 13 patients were evaluated at week 4 and the objective response rate ORR reached 100%. In all patients, there were 3 CR, 5 VGPR (including 1 Daratumumab-resistant patient), and 6 PR.

TABLE 2
Clinical response

| | | | Serum | | | | | | | | | | | | | | |
|---|---|---|---|---|---|---|---|---|---|---|---|---|---|---|---|---|---|
| | | | M | | | | | | | | | | | | | | |
| | | Fol- | pro- | | | | | | | | | | | | | | |
| Re- | | low | tien/ | Clinical response | | | | | | | | | | | | | |
| search | | up | sFLC | | | | | | | | | | | | | | Best |
| center | Patient ID | length | type | Baseline | | 2 w | | 4 w | | 8 w | | 12 w | | 16 w | 20 w | 6 m | ORR |
| Jiangsu | Z0203- | 6 m | IgG | 71.0 | g/L | 42.0 | g/L | 26.3 | g/L | 1.8 | g/L | 6.2 | g/L | 7.0 g/L | 16.4 g/L | 45 g/L | VGPR |
| Pro- | 00801C001 | | | | | | | | | UIF(−) | | UIF(−) | | UIF(−) | | | |
| vincial | Z0203- | 12 w | k | 3250 | mg/L | 3050 | mg/L | 75 | mg/L | 170 | mg/L | 960 | mg/L | PD | | | VGPR |
| Peo- | 00801C003 | | | | | | | SIF(−) | | | | | | | | | |
| ple's | | | | | | | | UIF(+) | | | | | | | | | |
| Hos- | | | | | | | | UPEP(−) | | | | | | | | | |
| pital | Z0203- | 6 m | IgG | 46.5 | g/L | 7.9 | g/L | 9.0 | g/L | SPEP(−) | | SPEP(−) | | SPEP(−) | SPEP(−) | 21.6 g/L | VGPR |
| | 00801C004 | | | | | | | | | UIF(−) | | UIF(−) | | UIF(−) | UIF(−) | | |
| | | | | | | | | | | SIF(+) | | SIF(+) | | SIF(+) | SIF(+) | | |
| | Z0203- | 20 w | IgG | 26.1 | g/L | 9.1 | g/L | 4.8 | g/L | 14.6 | g/L | 9 | g/L* | 10 g/L* | 17 g/L * | PD | PR |
| | 00801C008 | | | | | | | | | | | | | | | | |
| | Z0203- | 4 e | IgG | 55.7 | g/L | 45.4 | g/L | 23.3 | g/L | | | | | | | | PR |
| | 00801C010 | | | | | | | | | | | | | | | | |
| | Z0203- | 3 w | IgG | 22.5 | g/L | 18.8 | g/L | | | | | | | | | | SD |
| | 00801C011 | | | | | | | | | | | | | | | | |
| Tianjin | Z0203- | 8 w | IgA | 16.7 | g/L | 7.4 | g/L | 3.6 | g/L | SPEP(-) | | | | | | | VGPR |
| Hema- | 01301C001 | | | | | | | | | UIF(-) | | | | | | | |
| tology | | | | | | | | | | SIF(+) | | | | | | | |
| Hos- | Z0203- | 4 w | IgG | 50.2 | g/L | 27.1 | g/L | 15.0 | g/L | | | | | | | | PR |
| pital | 01301C003 | | | | | | | | | | | | | | | | |
| | Z0203- | 4 w | λ | 760 | mg/L | SPEP(−) | | SPEP(−) | | | | | | | | | VGPR |
| | 01301C006 | | | | | UIF(−) | | UIF(−) | | | | | | | | | |
| | | | | | | SIF(+) | | SIF(+) | | | | | | | | | |
| Peking | Z0203- | 4 w | IgG | 17.4 | g/L | 10.8 | g/L | 5.3 | g/L | | | | | | | | PR |
| Union | 00701C001 | | | | | | | | | | | | | | | | |
| Medical | Z0203- | 4 w | IgG | 21.7 | g/L | 15.8 | g/L | 7.9 | g/L | | | | | | | | PR |
| College | 00701C002 | | | | | | | | | | | | | | | | |
| Hos- | | | | | | | | | | | | | | | | | |
| pital | | | | | | | | | | | | | | | | | |
| Daupei | Z0203- | 12 w | λ | 187 | mg/L | 3.28 | mg/L | UIF- | | SIF- | | SIF- | | SIF- | SIF- | SIF- | sCR |
| Hos- | 00601C002 | | | | | | | SIF- | | UIF- | | UIF- | | UIF- | UIF- | UIF- | |
| pital | | | | | | | | MRD- | | MRD- | | MRD- | | MRD- | MRD- | MRD- | |
| | Z0203- | 4 w | κ | 209 | mg/L | SIF- | | SIF- | | SIF- | | SIF- | | | | | sCR |
| | 00601C004 | | | | | UIF- | | UIF- | | UIF- | | UIF- | | | | | |
| | | | | | | MRD- | | MRD- | | MRD- | | MRD- | | | | | |
| | Z0203- | 4 w | IgG | 28.27 | g/L | 24.3 | g/L | 8.6 | g/L | | | | | | | | PR |

| | | | | | | |
|---|---|---|---|---|---|---|
| 00601C005 | | | | | | |
| Z0203- | 2 W | κ | 121 | mg/L | SIF-UIF- | sCR |
| 00601C006 | | | | | MRD- | |

The treatment-emerging adverse events are shown in Table 3. Only one patient occurred grade 3 cytokine release syndrome in 15 patients. No neurotoxicity events and no dose-limiting toxicity (DLTs) were observed in the dose escalation. The cytopenias is mostly related to Cy/Flu lymphodepletion. It should be noted that the occurrence of a certain degree of cytokine release syndrome after treatment also illustrates the effectiveness of CART treatment from the side. None of the 15 patients had particularly serious cytokines, and C-CAR088 has better safety.

TABLE 3
Treatment-Emerging Adverse Events

| Treatment-Emerging Adverse Events | n (%) Overall | n (%) Grade 3/4 |
|---|---|---|
| Cytokine release syndrome | 14(93) | 1(7) |
| Neutropenia | 15(100) | 15(100) |
| Thrombocytopenia | 13(87) | 11(73) |
| Anemia | 12(80) | 6(40) |
| Increased AST | 5(33) | 3(20) |
| Infection | 3(20) | 3(20) |

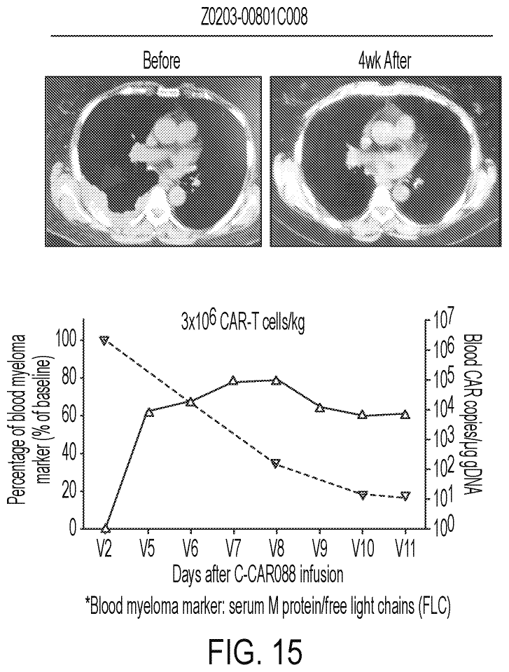

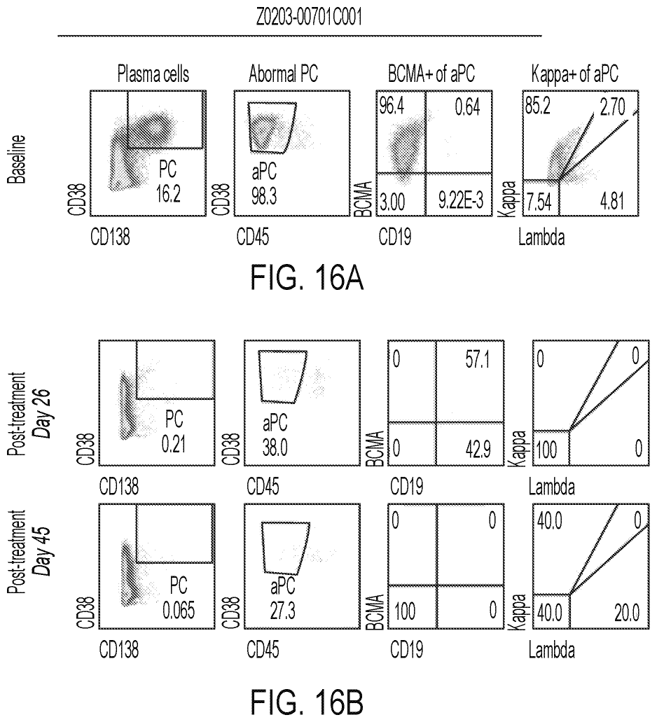

FIG. 15 shows the treatment condition of the patient of ID Z0203-00801C008. FIG. 16 shows the treatment condition of patient of ID Z0203-00701C001. The PET-CT images of the cancer lesions for one patient show that abnormal plasma cells in the bone marrow were significantly decreased after the C-CAR088 treatment (FIG. 15). FIGS. 16A and 16B show that majority of the PCs were abnormal (>90% were CD451o/-), were BCMA+ and clonal for Kappa light chain at the beginning of the experiment (Baseline). FIG. 16B shows that after 14 or 28 days of BCMA CAR-T treatment, abnormal PC in BM were significantly decreased, especially Kappa+PC, which decreased from the baseline level of 85.2% to 0%.

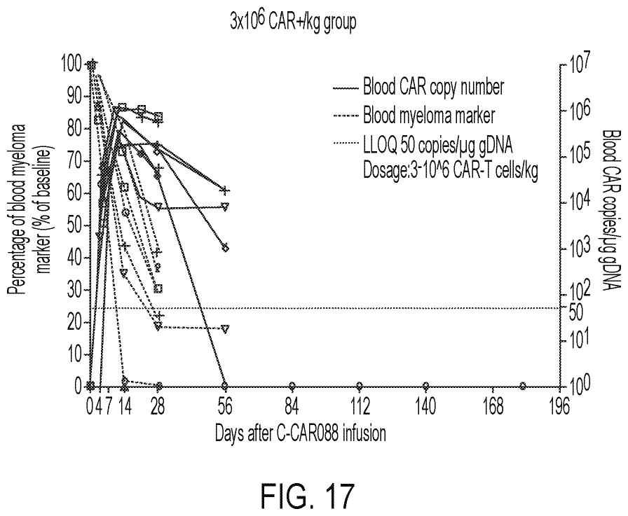

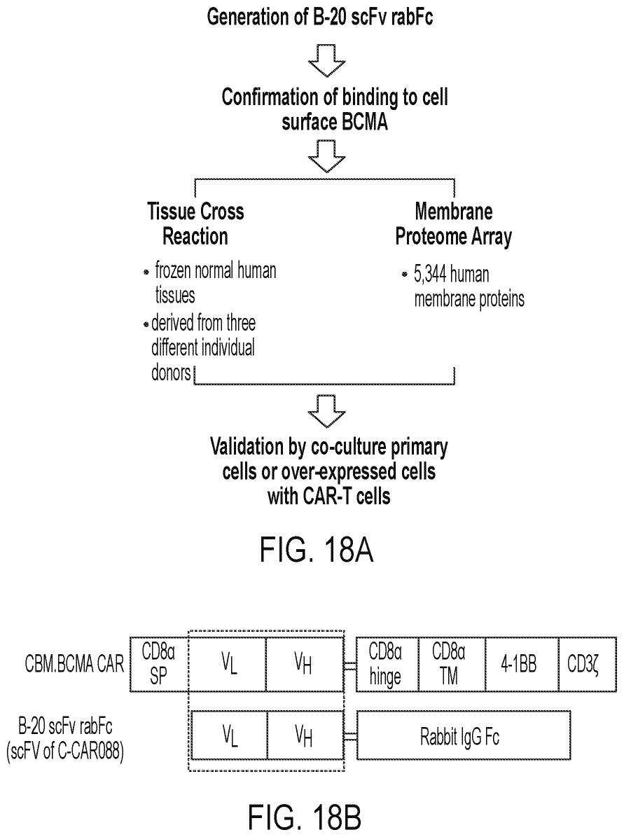

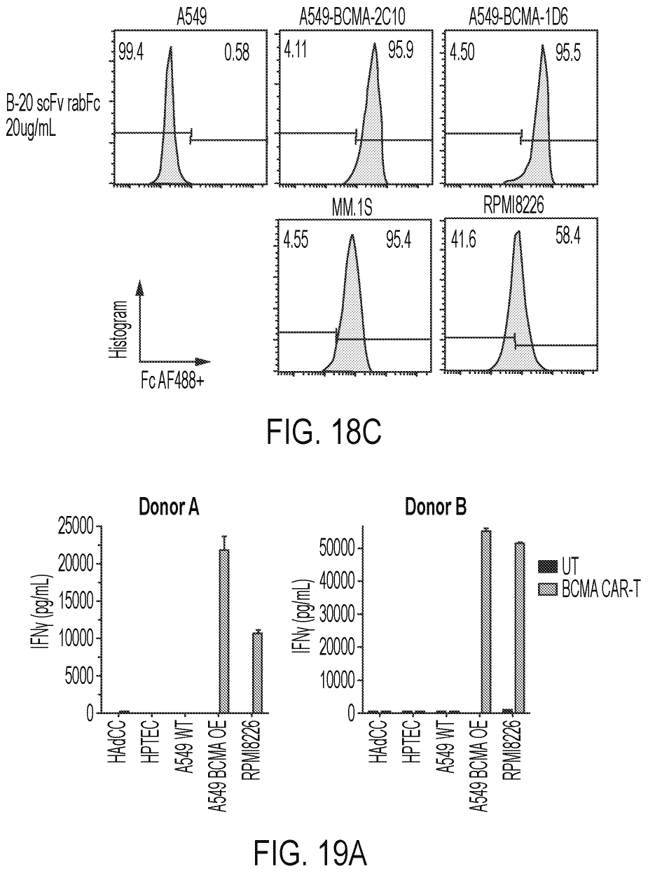

FIG. 17 shows the expansion of C-CAR088 and the decrease of M-protein/sFLC levels in the blood. The results showed that C-CAR088 cells expanded effectively after injection, and the level of M-protein/sFLC markers continued to decrease. The M-marker level of one patient was dropped to 0 on day 14.

Summary of observations for C-CAR088 was as follow.
In preclinical studies, C-CAR088 showed antitumor activity both in vitro and in vivo.
Early C-CAR088 trial results in patients with r/r MM support preclinical findings, showed promising efficacy and manageable safety profile.
The early clinical efficacy signal at low, suboptimal dose was encouraging.
Compared to KarMMa data, our current dose level from infused patients was well below the optimal dose of bb2121. 53% ( 8/15) were recently dosed (at −4 weeks).
Dose dependence was observed based on PK profile. C-CAR088 was well tolerated in patients.

### Example 12 an Anti-BCMA CAR T-Cell Therapy (C-CAR088) Showed Promising Safety and Efficacy Profile in Treating Relapsed or Refractory Multiple Myeloma (r/r MM)

Our studies demonstrated that C-CAR088 was considerably more cytotoxic towards tumor cells both in vitro and in vivo, compared to other anti-BCMA CARs.

We conducted a clinical trial of an anti-BCMA CAR (BCMA-20, also termed “C-CAR088”) in treating relapsed/refractory multiple myeloma (R/R MM) in patients.

Clinical trials are evaluated along a number of different criteria. Two key measures are overall response rate (ORR) and complete response rate (CR). When compared with other anti-BCMA CARs, the CARs of the claimed method offer better therapeutic efficacy in a clinical trial, as reflected by high response rates (95% overall response rate or ORR, and 67% complete response rate or CR) in treating relapsed/refractory multiple myeloma (R/R MM). Even when compared with JNJ-4528 (CARTITUDE-1), the percentage of adverse events, such as neurotoxicity, in the C-CAR088 was significantly lower. Specifically, for C-CAR088, only 4% patients experienced grade 1 neurotoxicity which resolved spontaneously. Thus, the CAR-T cells of the claimed method offered a favorable safety profile.

C-CAR088 demonstrated a manageable safety profile.
Most cases of CRS were grade ½ with median time to onset of 6 days.
Neurotoxicity (ICANS) was infrequent and generally low-grade with 1 grade 1 event.

Dose dependent responses occurred early and deepened.
ORR: 95.7%, with 43.5% CR rate at median time to CR: 1.6 months (range: 0.5-9.5).

Median time to response: 0.5 month.
At median 6.2 months follow up, 65.1% patients were progression-free at 6 months

In preclinical studies, C-CAR088 showed very good in vitro and in vivo anti-tumor activity and target specificity. Clinical trial results in 24 patients with r/r MM showed strong therapeutic index with promising efficacy and manageable safety profile.

We conducted a clinical trial of an anti-BCMA CAR (BCMA-20, also termed “C-CAR088”) in treating relapsed/refractory multiple myeloma (R/R MM) in patients. The patients' baseline demographics and clinical characteristics prior to the start of our anti-BCMA CAR treatment are shown in Table 4.

The median age of the patients dosed was 60 years (range: 45-74 years). The median number of prior lines of therapy was 4 (ranging from 2-12 prior therapies). All patients had received prior treatment with IMiDs (immunomodulatory drug) and proteasome inhibitors. 25% patents were previously treated with anti-CD38 monoclonal antibody, while 25% patents had received autologous hematopoietic stem cell transplant.

TABLE 4
Summary of baseline clinical characteristics of the patients

| Characteristic | N = 24 | Characteristic | N = 24 |
|---|---|---|---|
| Median age, yrs (range) | 60 (45-74) | High Risk Cytogenetics*, n (%) | |
| Age ≥ 65, n (%) | 9 (37.5) | 0 | 3 (12.5) |
| Male/Female, n | 14/10 | 1 | 5 (20.8) |
| MM subtype, n (%) | | 2 | 11 (45.8) |
| IgG | 14 (58.3) | 3 | 2 (8.3) |
| IgA | 3 (12.5) | Unknown | 3 (12.5) |
| IgD | 1 (4.2) | Median number of prior lines of therapy, n | 4 (2-12) |
| Light Chain | 6 (25) | (range) | |
| ECOG PS, n (%) | | Prior regimens, n (%) | |
| 0 | 15 (62.5) | Prior PIs and IMiDs | 24 (100) |
| 1 | 9 (37.5) | Prior ASCT | 6 (25.0) |
| ISS stage, n (%) | | Prior CD38 mAb | 6 (25.0) |
| I | 7 (29.2) | Received bridging therapy, n (%) | 4 (16.7) |
| II | 11 (45.8) | | |
| III | 6 (25) | | |

*Including 1q21 gain, del (17p), t (4; 15) and t (14; 16).

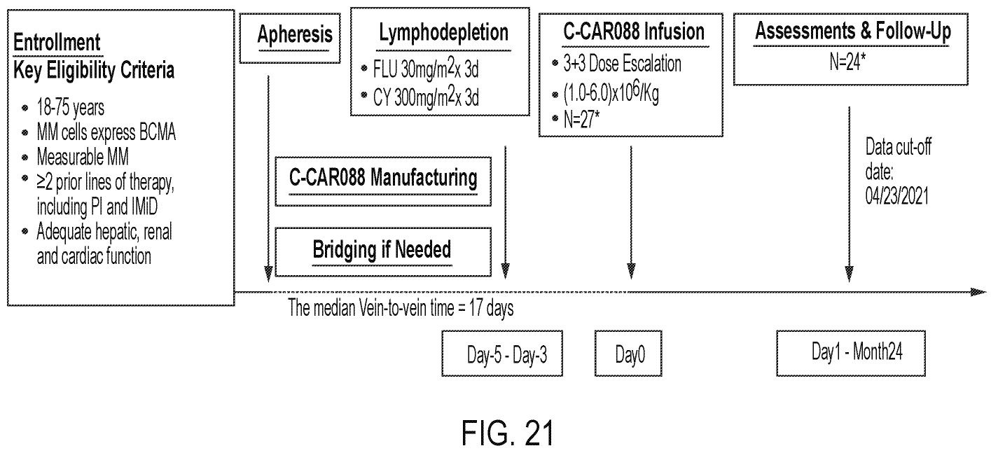

The clinical protocol, as well as the key inclusion criteria, is shown in FIG. 21. Specifically, qualified subjects were enrolled, and the collected peripheral blood leukocytes were used to produce the CAR-T cells (C-CAR088). The CAR-T cells were then frozen and stored below −135° C. until use. For CAR-T treatment, the CAR-T cells were thawed, and administration completed within 30-45 minutes.

At −5 and −3 days before the CAR-T infusion, the patients received lymphodepletion pretreatment, including fludarabine (30 mg/m²/d, intravenous, once per day for three days), and cyclophosphamide (300 mg/m²/d, intravenous, once per day for three days).

Approximately 72 hours after lymphodepletion, the patients were administered 1.0-6.0×10⁶ CAR-T cells/kg on day 0 as 3+3 dose escalation. Follow-ups with the patients were carried out after the infusion starting on day 1 through month 24.

The primary objectives included safety: rated of dose limiting toxicities; incidence and severity of treatment-emergent adverse events (CTCAEV5.0). Secondary objectives included efficacy: IMWG 2016 ORR; DOR; PFS; OS. Exploratory objectives included CAR-T expansion and persistence.

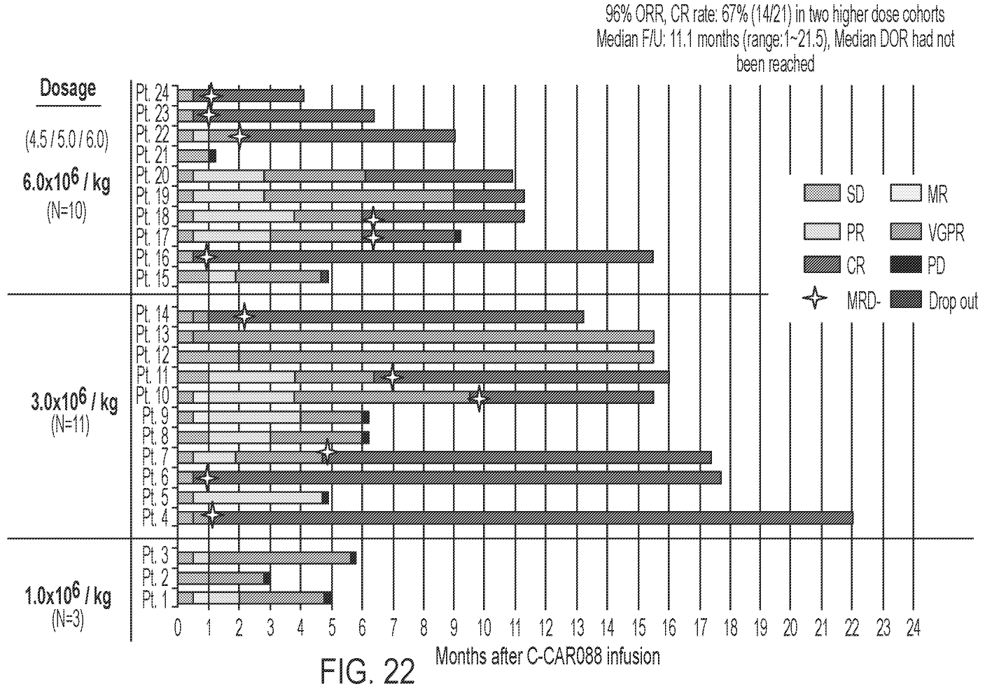

As shown in FIG. 22, an overall response rate (ORR, including CR and PR) of our anti-BCMA CAR-T trial is 96%. The best overall response (BOR) included 12 stringent complete responses (sCRs), 2 complete responses (CR), 8 very good partial responses (VGPRs) and 1 partial response (PR). The complete response (CR) is 67%. (SD: stable disease; PR: partial response; CR: complete response; PD: progressive disease; MR: minimal response; VGPR: very good partial response; MRD: minimal residual disease.)

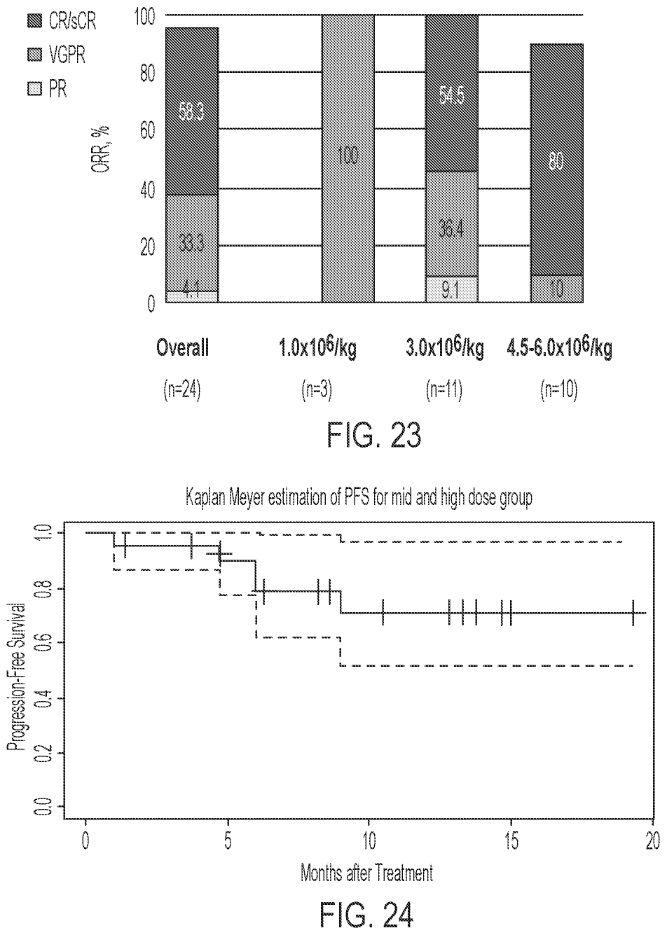

The CR/sCR, VGPR and PR for overall and each dose group are shown in Table 5 and FIG. 23. In the 3.0-6.0×10⁶ CAR-T cells/kg dose groups, 14/21 (66.7%) patients achieved a CR/sCR and all (14/14) patients achieved MRD negative by flow cytometry at 10⁻⁴-10⁻⁶.

TABLE 5

| | | 1.0 × 10⁶/kg | 3.0 × 10⁶/kg | 4.5-6.0 × 10⁶/kg |
|---|---|---|---|---|
| | Overall | (n = 3) | (n = 11) | (n = 10)* |
| Response | (n = 24) | (low dose) | (mid dose) | (high dose) |
| ORR, n (%) | 23 (95.8%) | 3 (100%) | 11 (100%) | 9 (90.0%) |
| CRR, n (%) | 13 (54.2%) | 0 | 6 (54.5%) | 7 (70.0%) |

*1 patient received 4.5 × 10⁶/kg and 1 received 5.0 × 10⁶/kg.

Table 6 below compares the C-CAR088 trial with those of Munshi et al. (KarMMa: Idecabtagene Vicleucel), Mailankody et al. (EVOLVE: Orvacabtagene Autoleucel) and Madduri et al. (CARTITUDE-1: JNJ-4528).

TABLE 6
Comparison of C-CAR088 clinical trial with other BCMA-specific CARs

| | KarMMa: | EVOLVE: | | |
|---|---|---|---|---|
| | Idecabtagene | Orvacabtagene | CARTITUDE-1: | |
| | Vicleucel | Autoleucel | JNJ-4528 | C-CAR088 |
| | (n = 128) | (n = 62) | (n = 97) | (n = 24) |
| Median age, yrs (range) | 61 (33-78) | 61 (33-77) | 61 (43-78) | 60 (45-74) |
| High risk cytogenetics | 35% | 41%* | 24% | 75%* |
| Tumor burden in | >50% PC: | — | >60% PC: | >50% PC: |
| BM, % | 51 | | 22 | 17 |
| Extramedullary PCs | 39% | 23% | 13% | 8% |
| Median prior lines of | 6 (3-16) | 6 (3-18) | 6 (3-18) | 4 (2-12) |
| therapies, No. (range) | | | | |
| Triple refractory, % | 84% | 94% | 88% | 25% |
| Bridging therapy | 88% | 63% | — | 17% |
| CRS: all/grade ≥ 3 | 84%/6% | 89%/3% | 95%/5% | 92%/4% |
| ICANS: all/grade ≥ 3 | 17%/3% | 13%/3% | 21%/10% | 4%/0 |
| | | | (Neurotoxicity) | |
| ORR | 73% | 92% | 97% | 95% |
| SCR/CR | 33% | 36% | 67% | 67% |
| Evaluable patients | 94% | 84% | 55% | 83% |
| with MRD neg ≥ 10⁵ | | | | |
| PFS, mos | 8.8 | 9.3⁺ | 12-mo PFS: | 6-mo PFS: |
| | | | 76.6% | 78.7%^{#} |

*Includes +1q21.
⁺PFS in lowest dose cohort (300 × 10⁶ cells).
^{#}Results in 3-6 × 10⁶ CAR-T cell/kg dose cohort.
ICANS: immune effector cell-associated neurotoxicity syndrome.

See, Munshi et al., Idecabtagene vicleucel (ide-cel; bb2121), a BCMA-targeted CAR T-cell therapy, in patients with relapsed and refractory multiple myeloma (RRMM): Initial KarMMa results, Journal of Clinical Oncology, 2020, 38(15) suppl., Abstract 8503; Mailankody et al., Orvacabtagene autoleucel (orva-cel), a B-cell maturation antigen (BCMA)-directed CAR T cell therapy for patients (pts) with relapsed/refractory multiple myeloma (RRMM): update of the phase ½ EVOLVE study (NCT03430011), Journal of Clinical Oncology, 2020, 38(15) suppl., Abstract 8504; Madduri et al., CARTITUDE-1: Phase 1b/2 Study of Ciltacabtagene Autoleucel, a B-Cell Maturation Antigen—Directed Chimeric Antigen Receptor T Cell Therapy, in Relapsed/Refractory Multiple Myeloma, 62nd ASH Annual Meeting and Exposition, Dec. 5-8, 2020, Abstract 177.

For our clinical trial of C-CAR088, the Kaplan Meier progression-free survival (PFS) estimates include a 6-month PFS of 78.7% for the mid- and high-dose group, with 95% confidence intervals (CIs) of 62.1%-99.7%. Median duration of response (DOR) was not reached. See Table 7 and FIG. 24.

TABLE 7

| | Time (month) | | | | | | | | | | | | | | | | | | |
|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 |
| n.risk | 21 | 19 | 19 | 18 | 16 | 16 | 13 | 13 | 10 | 9 | 8 | 8 | 5 | 3 | 2 | 1 | 1 | 1 | 1 |
| n.event | 1 | 0 | 0 | 0 | 1 | 2 | 0 | 0 | 1 | 0 | 0 | 0 | 0 | 0 | 0 | 0 | 0 | 0 | 0 |

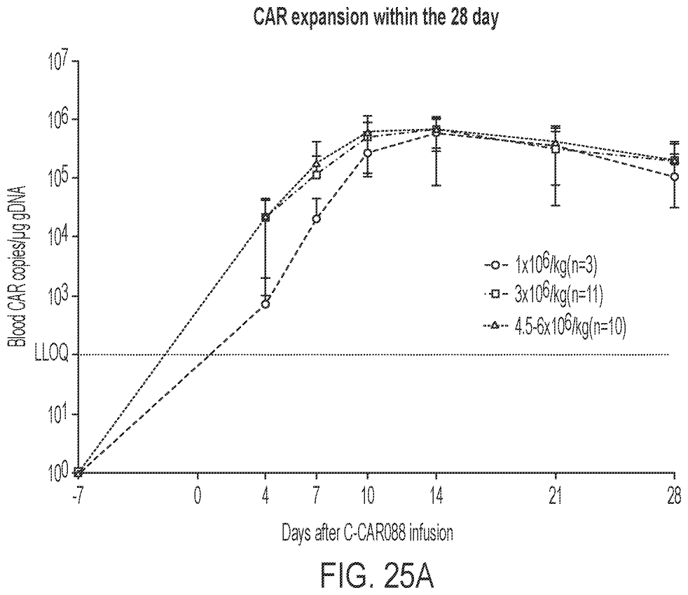

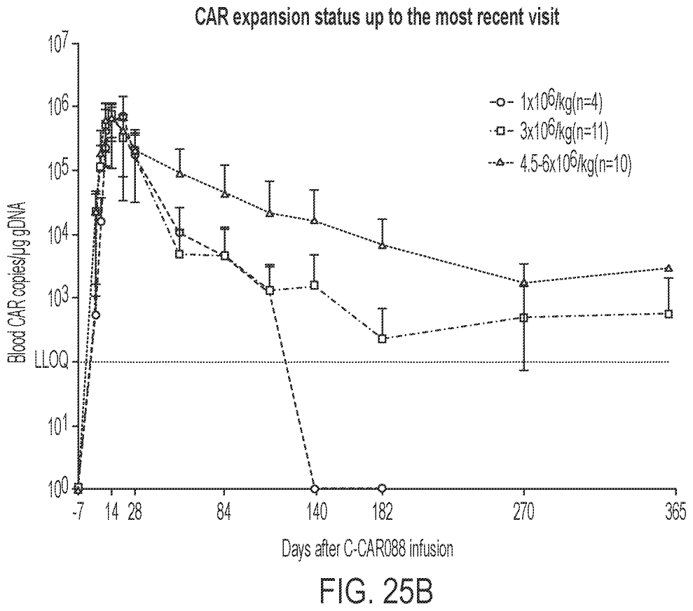

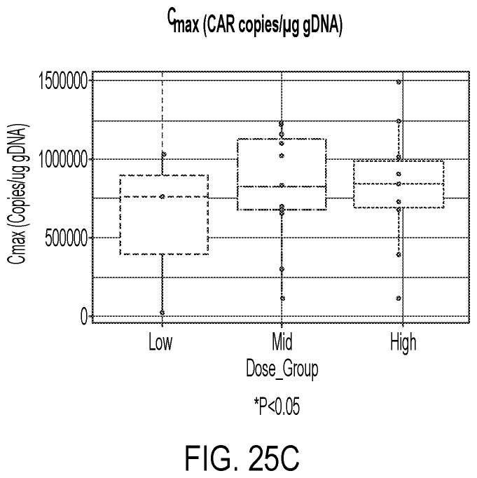

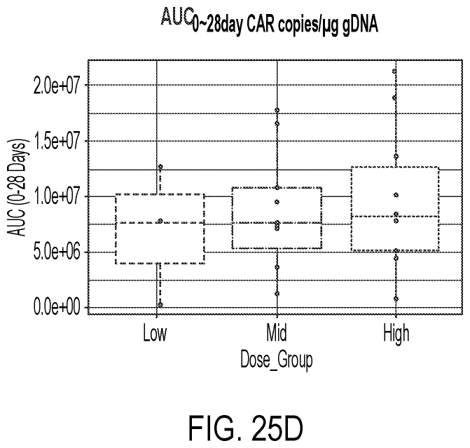

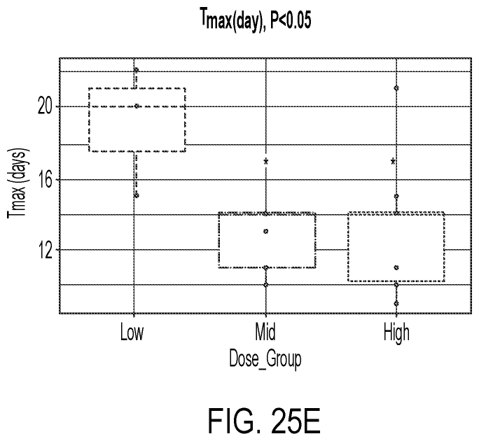

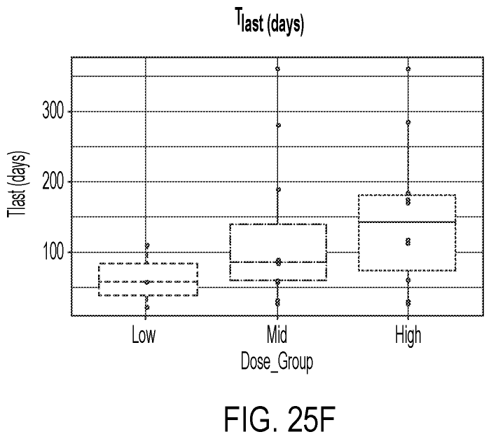

The time course of the CAR copies in the blood of the patients is shown in FIGS. 25A and 25B. Thus, the CAR levels were maintained in the blood after administration.

C-CAR088 treatment was well tolerated. The patients' adverse reactions (adverse events, AEs) were recorded (Tables 8 and 9). There was only 1 (4.2%) grade ≥3 cytokine release syndrome (CRS). Neurotoxicity was observed only in one patient (4.2%) which resolved spontaneously, with no grade ≥3 neurotoxicity. Cytopenia, such as neutropenia and thrombocytopenia, was mostly related to the fludarabine/cyclophosphamide (Cy/Flu) lymphodepletion. No dose-limiting toxicities were observed, and all adverse events were reversible. These demonstrated that our anti-BCMA CAR had an excellent safety profile.

TABLE 8

| | | Grade ≥ 3 |
|---|---|---|
| AEs (≥25%), n (%) | Any Grade (n = 24) | (n = 24) |
| Neutropenia | 24 (100%) | 22 (91.7%) |
| Lymphopenia | 22 (91.7%) | 20 (83.3%) |
| Thrombocytopenia | 22 (91.7%) | 9 (37.5%) |
| Anemia | 20 (83.3%) | 12 (50.0%) |
| Elevated AST/ALT | 13 (54.2%)/8 (33.3%) | 4 (16.7%)/0 (0) |
| Infection | 12 (50.0%) | 6 (25.0%) |

TABLE 9

| | | Grade ≥ 3 |
|---|---|---|
| CRS & Neurotoxicity * | Any Grade (n = 24) | (n = 24) |
| CRS, n (%) | 22 (91.7%) | 1 (4.2%) |
| Median days to onset, d (range) | 6 (1-11) | |
| Median days to resolution, d (range) | 5 (2-9) | |
| Treated with Tocilizumab, n (%) | 6 (26.1) | |
| Treated with Steroids, n (%) | 2 (8.7) | |
| Neurotoxicity, n (%) | 1 (4.2%) | 0 (0) |

| | |
|---|---|
| Days to onset, d | 8 |
| Days to resolution, d | 1 |

* ASTCT Consensus Grading for Cytokine Release Syndrome and Neurologic Toxicity Associated with Immune Effector Cells 2019.
CRS: Cytokine release syndrome

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A chimeric antigen receptor (CAR), comprising: an anti-BCMA antigen-binding region which comprises a light chain variable region (V_{L}) and a heavy chain variable region (V_{H}), V_{L} comprising three complementarity determining regions (CDRs), LCDR1, LCDR2 and LCDR3, V_{H} comprising three CDRs, HCDR1, HCDR2 and HCDR3,
(a) wherein LCDR1, LCDR2 and LCDR3 have amino acid sequences about 80% to about 100% identical to the amino acid sequences set forth in SEQ ID NO: 17, SEQ ID NO: 19, SEQ ID NO: 21, respectively, wherein HCDR1, HCDR2 and HCDR3 have amino acid sequences about 80% to about 100% identical to the amino acid sequences set forth in SEQ ID NO: 24, SEQ ID NO: 26, SEQ ID NO: 28, respectively;
(b) wherein LCDR1, LCDR2 and LCDR3 have amino acid sequences about 80% to about 100% identical to the amino acid sequences set forth in SEQ ID NO: 31, SEQ ID NO: 33, SEQ ID NO: 35, respectively, wherein HCDR1, HCDR2 and HCDR3 have amino acid sequences about 80% to about 100% identical to the amino acid sequences set forth in SEQ ID NO: 38, SEQ ID NO: 40, SEQ ID NO: 42, respectively; or
(c) wherein LCDR1, LCDR2 and LCDR3 have amino acid sequences about 80% to about 100% identical to the amino acid sequences set forth in SEQ ID NO: 45, SEQ ID NO: 47, SEQ ID NO: 49, respectively, wherein HCDR1, HCDR2 and HCDR3 have amino acid sequences about 80% to about 100% identical to the amino acid sequences set forth in SEQ ID NO: 52, SEQ ID NO: 54, SEQ ID NO: 56, respectively.

2. The CAR of claim 1, wherein V_{L} is located at the N-terminus of V_{H}.

3. The CAR of claim 1, wherein V_{L} and V_{H} have amino acid sequences about 80% to about 100% identical to amino acid sequences set forth in (a) SEQ ID NO: 1 and SEQ ID NO: 2, respectively; (a) SEQ ID NO: 3 and SEQ ID NO: 4, respectively; or (a) SEQ ID NO: 5 and SEQ ID NO: 6, respectively.

4. The CAR of claim 1, wherein the anti-BCMA antigen-binding region is a single-chain variable fragment (scFv) that specifically binds BCMA.

5. The CAR of claim 1, wherein the CAR further comprises one or more of the following:
(a) a signal peptide,
(b) a hinge region,
(c) a transmembrane domain,
(d) a co-stimulatory region, and
(e) a cytoplasmic signaling domain.

6. The CAR of claim 5, wherein the co-stimulatory region comprises a co-stimulatory region of 4-1BB (CD137), CD28, OX40, CD2, CD7, CD27, CD30, CD40, CD70, CD134, PD1, Dap10, CDS, ICAM-1, LFA-1 (CD11a/CD18), ICOS (CD278), NKG2D, GITR, TLR2, or combinations thereof.

7. The CAR of claim 5, wherein the cytoplasmic signaling domain comprises a cytoplasmic signaling domain of CD3 ζ.

8. The CAR of claim 5, wherein the hinge region comprises a hinge region of CD8, CD28, CD137, Ig4, or combinations thereof.

9. The CAR of claim 5, wherein the transmembrane domain comprises a transmembrane domain of CD8, CD28, CD3c, CD45, CD4, CD5, CD9, CD16, CD22, CD33, CD37, CD64, CD80, CD86, CD134, CD137, CD154, or combinations thereof.

10. An immune cell expressing the CAR of claim 1.

11. The immune cell of claim 10, wherein the immune cell is a T cell or a natural killer (NK) cell.

12. A nucleic acid encoding the CAR of claim 1.

13. A vector comprising the nucleic acid of claim 12.

14. A method of treating cancer, the method comprising administering the immune cell of claim 10 to a subject in need thereof.

15. The method of claim 14, wherein the cancer is a hematologic cancer.

16. The method of claim 14, wherein the cancer is a plasma-cell malignancy.

17. The method of claim 14, wherein the cancer is a BCMA-positive malignancy.

18. The method of claim 14, wherein the cancer is multiple myeloma (MM), or plasma cell leukemia.

19. The method of claim 14, wherein the immune cell is administered by infusion, injection, transfusion, implantation, and/or transplantation.

20. The method of claim 14, wherein the immune cell is administered intravenously, subcutaneously, intradermally, intranodally, intratumorally, intramedullary, intramuscularly, or intraperitoneally.

21. (canceled)

22. The method of claim 14, wherein the immune cell is allogeneic or autologous.

23. The method of claim 14, wherein the subject is a human.

24-33. (canceled)

34. The CAR of claim 1, comprising an amino acid sequence about 80% to about 100% identical to the amino acid sequence set forth in SEQ ID NO: 59, SEQ ID NO: 61, or SEQ ID NO: 63.


SEQUENCE LISTING (63 sequences)

SEQ ID NO: 1 - protein, 107 aa, Artificial Sequence
  BCMA-20 light chain variable region
DIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 2 - protein, 121 aa, Artificial Sequence
  BCMA-20 heavy chain variable region
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSS

SEQ ID NO: 3 - protein, 108 aa, Artificial Sequence
  BCMA-CA8 light chain variable region
DIQLTQTTSSLSASLGDRVTISCSASTTTSNYLNWYQQKPDGTVELVIYYTSNLHGGGPSRFSGSGSGTDYSLTIGYLEPEDVATYYCQQYRKLPWTFGGGSKLEIKR

SEQ ID NO: 4 - protein, 121 aa, Artificial Sequence
  BCMA-CA8 heavy chain variable region
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSS

SEQ ID NO: 5 - protein, 108 aa, Artificial Sequence
  BCMA-MO6 light chain variable region
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 6 - protein, 121 aa, Artificial Sequence
  BCMA-MO6 heavy chain variable region
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 7 - protein, 111 aa, Artificial Sequence
  BCMA-1 light chain variable region
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 8 - protein, 117 aa, Artificial Sequence
  BCMA-1 heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 9 - protein, 21 aa, Artificial Sequence
  CD8 Signal peptide
MALPVTALLLPLALLLHAARP

SEQ ID NO: 10 - protein, 18 aa, Artificial Sequence
  linker
GSTSGSGKPGSGEGSTKG

SEQ ID NO: 11 - protein, 15 aa, Artificial Sequence
  linker
GGGGSGGGGSGGGGS

SEQ ID NO: 12 - protein, 55 aa, Artificial Sequence
  Hinge
FVPVFLPAKPTTTPAPRPPTPAPTIASQPLSLRPEACRPAAGGAVHTRGLDFACD

SEQ ID NO: 13 - protein, 24 aa, Artificial Sequence
  CD8 transmembrane domain
IYIWAPLAGTCGVLLLSLVITLYC

SEQ ID NO: 14 - protein, 42 aa, Artificial Sequence
  4-1BB Costimulatory sequence
KRGRKKLLYIFKQPFMRPVQTTQEEDGCSCRFPEEEEGGCEL

SEQ ID NO: 15 - protein, 113 aa, Artificial Sequence
  CD3 zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 23 aa, Artificial Sequence
  synthetic
DIQMTQSPSSLSASVGDRVTITC

SEQ ID NO: 17 - protein, 11 aa, Artificial Sequence
  synthetic
RASQGISNYLN

SEQ ID NO: 18 - protein, 15 aa, Artificial Sequence
  synthetic
WYQQKPGKAPKPLIY

SEQ ID NO: 19 - protein, 7 aa, Artificial Sequence
  synthetic
YTSNLQS

SEQ ID NO: 20 - protein, 32 aa, Artificial Sequence
  synthetic
GVPSRFSGSGSGTDYTLTISSLQPEDFATYYC

SEQ ID NO: 21 - protein, 9 aa, Artificial Sequence
  synthetic
MGQTISSYT

SEQ ID NO: 22 - protein, 9 aa, Artificial Sequence
  synthetic
FGQGTKLEI

SEQ ID NO: 23 - protein, 30 aa, Artificial Sequence
  synthetic
EVQLVESGGGLVQPGGSLRLSCAASGFTFS

SEQ ID NO: 24 - protein, 5 aa, Artificial Sequence
  synthetic
NFDMA

SEQ ID NO: 25 - protein, 14 aa, Artificial Sequence
  synthetic
WVRQAPGKGLVWVS

SEQ ID NO: 26 - protein, 17 aa, Artificial Sequence
  synthetic
SITTGADHAIYADSVKG

SEQ ID NO: 27 - protein, 32 aa, Artificial Sequence
  synthetic
RFTISRDNAKNTLYLQMNSLRAEDTAVYYCVR

SEQ ID NO: 28 - protein, 12 aa, Artificial Sequence
  synthetic
HGYYDGYHLFDY

SEQ ID NO: 29 - protein, 11 aa, Artificial Sequence
  synthetic
WGQGTLVTVSS

SEQ ID NO: 30 - protein, 23 aa, Artificial Sequence
  synthetic
DIQLTQTTSSLSASLGDRVTISC

SEQ ID NO: 31 - protein, 11 aa, Artificial Sequence
  synthetic
SASTTTSNYLN

SEQ ID NO: 32 - protein, 15 aa, Artificial Sequence
  synthetic
WYQQKPDGTVELVIY

SEQ ID NO: 33 - protein, 7 aa, Artificial Sequence
  synthetic
YTSNLHG

SEQ ID NO: 34 - protein, 32 aa, Artificial Sequence
  synthetic
GGPSRFSGSGSGTDYSLTIGYLEPEDVATYYC

SEQ ID NO: 35 - protein, 9 aa, Artificial Sequence
  synthetic
QQYRKLPWT

SEQ ID NO: 36 - protein, 11 aa, Artificial Sequence
  synthetic
FGGGSKLEIKR

SEQ ID NO: 37 - protein, 30 aa, Artificial Sequence
  synthetic
EVQLQQSGAVLARPGASVKMSCKGSGYTFT

SEQ ID NO: 38 - protein, 5 aa, Artificial Sequence
  synthetic
NYWMH

SEQ ID NO: 39 - protein, 14 aa, Artificial Sequence
  synthetic
WVKQRPGQGLEWIG

SEQ ID NO: 40 - protein, 17 aa, Artificial Sequence
  synthetic
ATYRGHSDTYYNQKFKG

SEQ ID NO: 41 - protein, 32 aa, Artificial Sequence
  synthetic
KAKLTAVTSTSTAYMELSSLTNEDSAVYYCTR

SEQ ID NO: 42 - protein, 12 aa, Artificial Sequence
  synthetic
GAIYNGYDVLDN

SEQ ID NO: 43 - protein, 11 aa, Artificial Sequence
  synthetic
WGQGTLVTVSS

SEQ ID NO: 44 - protein, 23 aa, Artificial Sequence
  synthetic
DIQMTQSPSSLSASVGDRVTITC

SEQ ID NO: 45 - protein, 11 aa, Artificial Sequence
  synthetic
SASQDISNYLN

SEQ ID NO: 46 - protein, 15 aa, Artificial Sequence
  synthetic
WYQQKPGKAPKLLIY

SEQ ID NO: 47 - protein, 7 aa, Artificial Sequence
  synthetic
YTSNLHS

SEQ ID NO: 48 - protein, 32 aa, Artificial Sequence
  synthetic
GVPSRFSGSGSGTDFTLTISSLQPEDFATYYC

SEQ ID NO: 49 - protein, 9 aa, Artificial Sequence
  synthetic
QQYRKLPWT

SEQ ID NO: 50 - protein, 11 aa, Artificial Sequence
  synthetic
FGQGTKLEIKR

SEQ ID NO: 51 - protein, 30 aa, Artificial Sequence
  synthetic
QVQLVQSGAEVKKPGSSVKVSCKASGGTFS

SEQ ID NO: 52 - protein, 5 aa, Artificial Sequence
  synthetic
NYWMH

SEQ ID NO: 53 - protein, 14 aa, Artificial Sequence
  synthetic
WVRQAPGQGLEWMG

SEQ ID NO: 54 - protein, 17 aa, Artificial Sequence
  synthetic
ATYRGHSDTYYNQKFKG

SEQ ID NO: 55 - protein, 32 aa, Artificial Sequence
  synthetic
RVTITADKSTSTAYMELSSLRSEDTAVYYCAR

SEQ ID NO: 56 - protein, 12 aa, Artificial Sequence
  synthetic
GAIYDGYDVLDN

SEQ ID NO: 57 - protein, 11 aa, Artificial Sequence
  synthetic
WGQGTLVTVSS

SEQ ID NO: 58 - DNA, 1494 bp, Artificial Sequence
  synthetic
atggccttaccagtgaccgccttgctcctgccgctggccttgctgctccacgccgccaggccggacatccagatgacccagtccccctcctccctgtccgcctccgtgggcgaccgggtgaccatcacctgccgggcctcccagggcatctccaactacctgaactggtaccagcagaagcccggcaaggcccccaagcccctgatctactacacctccaacctgcagtccggcgtgccctcccggttctccggctccggctccggcaccgactacaccctgaccatctcctccctgcagcccgaggacttcgccacctactactgcatgggccagaccatctcctcctacaccttcggccagggcaccaagctggagatcaagggtggcggtggctcgggcggtggtgggtcgggtggcggcggatctgaggtgcagctggtggagtccggcggcggcctggtgcagcccggcggctccctgcggctgtcctgcgccgcctccggcttcaccttctccaacttcgacatggcctgggtgcggcaggcccccggcaagggcctggtgtgggtgtcctccatcaccaccggcgccgaccacgccatctacgccgactccgtgaagggccggttcaccatctcccgggacaacgccaagaacaccctgtacctgcagatgaactccctgcgggccgaggacaccgccgtgtactactgcgtgcggcacggctactacgacggctaccacctgttcgactactggggccagggcaccctggtgaccgtgtcctccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 59 - protein, 497 aa, Artificial Sequence
  synthetic
MALPVTALLLPLALLLHAARPDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKGGGGSGGGGSGGGGSEVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 1497 bp, Artificial Sequence
  synthetic
atggccttaccagtgaccgccttgctcctgccgctggccttgctgctccacgccgccaggccggatatccagctgacccagaccacaagcagcctgagcgcctccctgggcgacagggtgaccattagctgcagcgccagccaggacatcagcaactacctgaactggtaccagcagaagcccgacggcaccgtggagctcgtgatctactacacctccaacctgcacagcggcgtgcccagcaggttctctggcagcggcagcggcaccgactacagcctgaccatcggctatctggagcccgaggacgtcgccacctactactgccagcagtacaggaagctgccctggaccttcggcggaggctctaagctggagattaagcgtggtggcggtggctcgggcggtggtgggtcgggtggcggcggatctgaggtgcagctgcagcagagcggcgccgtgctggccaggcccggagctagcgtgaagatgagctgcaagggcagcggctacaccttcaccaactactggatgcactgggtgaaacagaggcccggccagggactggagtggatcggcgccacctacaggggccacagcgacacctactacaaccagaagttcaagggcaaggccaagctgaccgccgtgacctcaaccagcaccgcctacatggaactgagcagcctgaccaacgaggacagcgccgtctattactgcaccaggggcgccatctacaacggctacgacgtgctggacaattggggccagggaacactagtgaccgtgtccagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 61 - protein, 498 aa, Artificial Sequence
  synthetic
MALPVTALLLPLALLLHAARPDIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKRGGGGSGGGGSGGGGSEVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1497 bp, Artificial Sequence
  synthetic
atggccttaccagtgaccgccttgctcctgccgctggccttgctgctccacgccgccaggccggacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgtggtggcggtggctcgggcggtggtgggtcgggtggcggcggatctcaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacgacggctacgacgtgctggacaactggggccagggcacactagtgaccgtgtccagcttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 63 - protein, 498 aa, Artificial Sequence
  synthetic
MALPVTALLLPLALLLHAARPDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR